(19) United States
(12) Patent Application Publication
(10) Pub. No.: US 2016/0215305 A1
(43) Pub. Date: Jul. 28, 2016

(54) Process of Fermenting A Lignocellulosic Biomass

(71) Applicant: AUBURN UNIVERSITY, Auburn, AL (US)

(72) Inventors: Maobing TU, Cincinnati, OH (US); Rui XIE, Newbury Park, CA (US)

(21) Appl. No.: 15/006,524

(22) Filed: Jan. 26, 2016

Related U.S. Application Data
(60) Provisional application No. 62/107,791, filed on Jan. 26, 2015.

(51) Int. Cl. C12P 7/10

(52) U.S. Cl. CPC C12P 7/10; C12P 2201/00

(57) ABSTRACT

The present disclosure provides methods of fermenting a lignocellulosic biomass, wherein a nucleophile is added to deactivate carbonyl-containing fermentation inhibitors in hydrolysate formulated by pretreating the lignocellulosic biomass. The disclosure also provides methods of fermenting a lignocellulosic biomass, wherein a nucleophile is added to deactivate carbonyl-containing fermentation inhibitors in a slurry formulated by pretreating the lignocellulosic biomass.

### CROSS-REFERENCE TO RELATED APPLICATIONS

This application claims the benefit under 35 USC §119(e) of U.S. Provisional Application Ser. No. 62/107,791, filed on Jan. 26, 2015, the entire disclosure of which is incorporated herein by reference.

### TECHNICAL FIELD

The invention relates to the fermentation of a lignocellulosic biomass with nucleophile to enhance alcoholic fermentation. The invention includes methods for treating a hydrolysate or a slurry obtained from lignocellulosic biomass with a nucleophile, such as nucleophilic amino acids, wherein the nucleophile can deactivate carbonyl-containing fermentation inhibitors in the hydrolysate or slurry.

### BACKGROUND AND SUMMARY OF THE INVENTION

The production of biofuels from lignocellulosic biomass using a biochemical conversion process offers great promise to reduce the dependence of the world on petroleum based fuels. In order to be successful, the biomass typically needs to be pretreated to break down its recalcitrance, which is subsequently hydrolyzed to monomeric sugars using hydrolytic enzymes and fermented to biofuels by microorganisms. However, the pretreatment of biomass generates a wide range of toxic compounds from the degradation of carbohydrates, lignin, and extractives, which significantly inhibits the microbial fermentation.

Substantial studies have concentrated on identifying potential inhibitors using advanced analytical tools and developing detoxification methods to remove their inhibition. However, due to a large number of degradation compounds and low concentrations, inhibitors that contribute to the most potent inhibition in hydrolysates fermentation remain elusive. Consequently, without the correct targets, using current detoxification or conditioning methods known in the art are not cost effective and are not chemically selective.

Previous research has been developed regarding physical, biological, and chemical approaches to detoxify biomass hydrolysates. For example, evaporation and steam stripping have been used to remove the volatile compounds but not the non-volatile toxic compounds. Ligninolytic enzymes such as laccase and peroxidase are able to transform phenolic compounds through presumable oxidative polymerization, these methods require long treatment times (e.g., 12 hours or more) and high costs for preparation of the enzymes. Furthermore, bioabatement processes were a recent biological attempt to use microbes such as Coniochaeta ligniaria NRRL30616 to metabolize a wide range of inhibitors in dilute-acid biomass hydrolysates, which also required a long treatment time (e.g., 24 hours).

Although activated charcoal and anion exchange resins could adsorb inhibitors through physical interactions and desirably increased fermentability of hydrolysates, both methods cause considerable loss of sugars and increase the cost in bioconversion process. Alkaline treatment or overliming has been a widely-used method to detoxify biomass hydrolysates while the gypsum salts and precipitates produced during the detoxification could be significant issues for subsequent processes. However, failing to target on the most potent inhibitors instead of randomly removing the degradation compounds has proven to not be a cost effective approach to improve the fermentation of hydrolysates.

Therefore, there exists a need for new methods for improving the fermentation of hydrolysates in prompt and cost-efficient manner. Accordingly, the present disclosure provides improved methods for fermenting a lignocellulosic biomass using a biochemical conversion process.

The methods comprising fermentation of a lignocellulosic biomass according to the present disclosure provide several advantages compared to other methods known in the art. Importantly, correlating the inhibitory activity of toxic degradation compounds to their structural features assists to design a cost-effective detoxification approach for biomass hydrolysates. Aromatic aldehydes and ketones (syringaldehyde) are mostly degradation compounds of lignin and extractives. Among them, the aromatic aldehydes are a group of α, β-unsaturated compounds having a carbon-carbon double bond (C═C) conjugated to the carbonyl group (C═O), such as cinnamaldehyde and coniferyl aldehyde. Carbonyl compounds are electrophilic. Due to high electronegativity of oxygen relative to carbon, the carbon-oxygen double bond is polarized, creating a partially positive charge on the carbonyl carbon atom. The electron-poor carbonyl carbon could form covalent bonds with biological nucleophiles such as proteins and nucleic acids, leading to inhibition on protein functions, DNA duplication, or even loss of cell activity.

In the case of α, β-unsaturated aldehydes, the electronegative oxygen atom in the carbonyl group can also withdraw electrons from the β carbon, making it polarized and more electrophilic than a regular alkene carbon. Therefore, α, β-unsaturated aldehydes have two reactive functional groups that can participate individually or cooperatively in a series reactions with nucleophiles in the microbial cells. Consequently, inactivation of these electrophilic groups can be the key to remove the fermentation inhibition of carbonyl compounds.

The inhibitory effects of carbonyl compounds are controlled by their electrophilic functional groups, which can be detoxified by reacting with nucleophiles such as amino acids. Most of amino acids contain a primary amine group and important side chain functional group, such as thiol group in cysteine. These functional groups can react readily with electrophilic carbonyl compounds, thus detoxify these carbonyl compounds. Accordingly, the present disclosure provides that highly nucleophilic amino acids (e.g., cysteine) can to be used to detoxify biomass hydrolysates selectively and in an environmentally friendly manner because no extra waste is produced and additional amino acids can be readily consumed by microbes. In particular, the nucleophile can detoxify fermentation inhibitors (including carbonyl aldehydes, carbonyl ketones and carboxylic acids) in the hydrolysate or slurry to provide the advantages described herein.

The following numbered embodiments are contemplated and are non-limiting:

1. A method of fermenting a lignocellulosic biomass, the method comprising the steps of

pretreating the lignocellulosic biomass to provide a hydrolysate;

adding a nucleophile to the hydrolysate; and

adding a microorganism to the hydrolysate to produce an alcohol,

wherein a sufficient amount of the nucleophile is added to deactivate carbonyl-containing fermentation inhibitors in the hydrolysate.

2. The method of clause 1, wherein the hydrolysate is filtered into a solid fraction and a liquid fraction prior to addition of the nucleophile to the liquid fraction.

3. The method of clause 2, wherein the liquid fraction is concentrated prior to addition of the nucleophile.

4. The method of clause 3, wherein the pH of the concentrated liquid fraction is adjusted prior to addition of the nucleophile.

5. The method of any of clauses 1 to 4, wherein the pretreatment step increases the accessibility of celluloses to cellulosic enzymes.

6. The method any of clauses 1 to 5, wherein the pretreatment step comprises adding an organic solvent or an ionic liquid.

7. The method of any of clauses 1 to 6, wherein the pretreatment step comprises pretreating the lignocellulosic biomass with saturated steam.

8. The method of any of clauses 1 to 6, wherein the pretreatment step is selected from the group consisting of chemical pretreatment, steam explosion, organosolv pretreatment, ammonia fibre explosion (AFEX), ionic liquid pretreatment, and biological pretreatment.

9. The method of any of clauses 1 to 8, wherein the pretreatment step comprises adding an acid selected from the group consisting of sulfuric acid, phosphoric acid, nitric acid, hydrochloric acid, and combinations thereof.

10. The method of any of clauses 1 to 9, wherein the pretreatment step comprises adding a base selected from the group consisting of sodium hydroxide, calcium hydroxide, potassium hydroxide, ammonia, and combinations thereof.

11. The method of any of clauses 1 to 10, wherein the pretreatment step comprises addition of an acid.

12. The method of clause 11, wherein the pretreatment step comprises adding less than 5% w/w acid.

13. The method of clause 11, wherein the pretreatment step comprises adding about 1% w/w acid.

14. The method of any of clauses 1 to 13, wherein the lignocellulosic biomass is an agricultural biomass.

15. The method of any of clauses 1 to 14, wherein the lignocellulosic biomass is selected from the group consisting of corn, corn stover, corn cobs, wood chips, softwood wood chips, hardwood wood chips, wheat straw, rice straw, hybrid poplar, sugarcane bagasse, switchgrass, miscanthus, forest thinnings, forest residues, agricultural residues, and combinations thereof.

16. The method of any of clauses 1 to 14, wherein the lignocellulosic biomass is wood.

17. The method of any of clauses 1 to 16, wherein the pretreatment step comprises organosolv pulping.

18. The method of any of clauses 1 to 17, wherein the pretreatment step comprises disrupting the lignocellulosic matrix.

19. The method of any of clauses 1 to 18, wherein the pretreatment step comprises soaking in sulfuric acid.

20. The method of any of clauses 1 to 19, wherein the pretreatment step comprises soaking in sulfuric acid at an elevated temperature.

21. The method of any of clauses 1 to 20, wherein one or more hemicellulose sugars are recovered after the pretreatment step.

22. The method of any of clauses 1 to 21, wherein the pH of the hydrolysate is about 1.8.

23. The method of any of clauses 1 to 22, wherein the hydrolysate comprises sugars selected from the group consisting of glucose, mannose, xylose, galactose, arabinose, and combinations thereof.

24. The method of any of clauses 1 to 23, wherein the hydrolysate comprises sugar degradation compounds selected from the group consisting of acetic acid, levulinic acid, formic acid, furfural, hydroxymethylfurfural (HMF), vanillin, cinnamaldehyde, syringaldehyde, phenolic and aromatic compounds, and combinations thereof.

25. The method of any of clauses 1 to 24, wherein the hydrolysate is substantially not fermentable.

26. The method of any of clauses 2 to 25, wherein the pH of the liquid fraction is about 1.8.

27. The method of any of clauses 2 to 26, wherein the liquid fraction comprises sugars selected from the group consisting of glucose, mannose, xylose, galactose, arabinose, and combinations thereof.

28. The method of any of clauses 2 to 27, wherein the liquid fraction comprises sugar degradation compounds selected from the group consisting of acetic acid, levulinic acid, formic acid, furfural, hydroxymethylfurfural (HMF), vanillin, cinnamaldehyde, syringaldehyde, phenolic and aromatic compounds, and combinations thereof.

29. The method of any of clauses 2 to 28, further comprising the step of contacting the solid fraction with hydrolytic enzymes to provide monomeric sugars.

30. The method of any of clauses 1 to 29, wherein the fermentation inhibitor is a carbonyl-containing compound.

31. The method of any of clauses 1 to 29, wherein the fermentation inhibitor is a ketone or an aldehyde.

32. The method of any of clauses 1 to 29, wherein the fermentation inhibitor is an aromatic ketone or an aromatic aldehyde.

33. The method of any of clauses 1 to 29, wherein the fermentation inhibitor is an α,β-unsaturated ketone or an α,β-unsaturated aromatic aldehyde.

34. The method of any of clauses 1 to 33, wherein the addition of the nucleophile to the hydrolysate is performed prior to addition of the microorganism.

35. The method of any of clauses 1 to 34, wherein the nucleophile is added to the hydrolysate prior to addition of the microorganism.

36. The method of any of clauses 1 to 35, wherein the nucleophile is added to the hydrolysate wherein the hydrolysate is substantially free of the microorganism.

37. The method of any of clauses 1 to 36, wherein adding the hydrolysate with the nucleophile takes place at about 60° C. and at a pH of about 6.0 for about 2 hours.

38. The method of any of clauses 3 to 36, wherein adding the concentrated liquid fraction with the nucleophile takes place at about 60° C. and at a pH of about 6.0 for about 2 hours.

39. The method of any of clauses 1 to 38, wherein the nucleophile is an amino acid.

40. The method any of clauses 1 to 38, wherein the nucleophile is an amino acid selected from the group consisting of glycine, alanine, valine, leucine, isoleucine, proline, phenylalanine, tyrosine, tryptophan, serine, threonine, cysteine, methionine, asparagine, glutamine, lysine, histidine, arginine, aspartate, glutamate, and combinations thereof.

41. The method of any of clauses 1 to 38, wherein the nucleophile is an amino acid selected from the group consisting of cysteine, histidine, tryptophan, asparagine, lysine, and combinations thereof.

42. The method of any of clauses 1 to 38, wherein the nucleophile comprises cysteine, histidine, or a combination thereof.

43. The method of any of clauses 1 to 38, wherein the nucleophile is cysteine, histidine, or a combination thereof.

44. The method of any of clauses 1 to 38, wherein the nucleophile consists essentially of cysteine, histidine, or a combination thereof.

45. The method of any of clauses 1 to 38, wherein the nucleophile consists of cysteine, histidine, or a combination thereof.

46. The method of any of clauses 1 to 38, wherein the nucleophile is cysteine or histidine.

47. The method of any of clauses 1 to 38, wherein the nucleophile is cysteine.

48. The method of any of clauses 1 to 38, wherein the nucleophile consists essentially of cysteine.

49. The method of any of clauses 1 to 38, wherein the nucleophile consists of cysteine.

50. The method of any of clauses 47 to 49, wherein the concentration of cysteine is about 5.0 mM.

51. The method of any of clauses 1 to 38, wherein the nucleophile is histidine.

52. The method of any of clauses 1 to 38, wherein the nucleophile consists essentially of histidine.

53. The method of any of clauses 1 to 38, wherein the nucleophile consists of histidine.

54. The method of any of clauses 1 to 38, wherein the nucleophile is glycine.

55. The method of any of clauses 1 to 38, wherein the nucleophile consists essentially of glycine.

56. The method of any of clauses 1 to 38, wherein the nucleophile consists of glycine.

57. The method of any of clauses 1 to 56, wherein the hydrolysate is adjusted to a pH of about 6 before addition of the nucleophile.

58. The method of any of clauses 1 to 56, wherein the hydrolysate is adjusted to a pH of about 6 before addition of the nucleophile.

59. The method of any of clauses 2 to 56, wherein the liquid fraction is adjusted to a pH of about 6 after addition of the nucleophile.

60. The method of any of clauses 2 to 56, wherein the liquid fraction is adjusted to a pH of about 6 after addition of the nucleophile.

61. The method of any of clauses 1 to 60, wherein the microorganism is a yeast.

62. The method of any of clauses 1 to 60, wherein the microorganism is Saccharomyces cerevisiae.

63. The method of any of clauses 1 to 60, wherein the microorganism is a bacteria.

64. The method of any of clauses 1 to 60, wherein the microorganism is E Coli.

65. The method of any of clauses 1 to 60, wherein the microorganism is Zymomonas mobilis.

66. The method of any of clauses 1 to 60, wherein the microorganism is Clostridium sp.

67. The method of any of clauses 1 to 60, wherein the microorganism is Clostridium acetobutylicum.

68. The method of any of clauses 1 to 67, wherein the hydrolysate is adjusted to a pH of about 6 with NaOH or H₂SO₄ and sterilized by passing 0.2 μm sterile filters.

69. The method of any of clauses 1 to 68, wherein the nucleophile prevents carbonyl compounds released during the biomass pretreatments from inhibiting biomass hydrolysates fermentation.

70. The method of any of clauses 1 to 69, wherein the nucleophile has a nucleophilicity parameter (N) of about 10 or greater.

71. The method of any of clauses 1 to 69, wherein the nucleophile has a nucleophilicity parameter (N) of about 20 or greater.

72. The method of any of clauses 1 to 71, wherein the addition of the nucleophile to the hydrolysate is performed at a temperature of about 50° C. to about 100° C.

73. The method of any of clauses 1 to 71, wherein the addition of the nucleophile to the hydrolysate is performed at a temperature of about 50° C. to about 90° C.

74. The method of any of clauses 1 to 71, wherein the addition of the nucleophile to the hydrolysate is performed at a temperature of about 60° C. to about 80° C.

75. The method of any of clauses 1 to 71, wherein the addition of the nucleophile to the hydrolysate is performed at a temperature of about 70° C. to about 80° C.

76. The method of any of clauses 1 to 75, wherein the addition of the nucleophile to the hydrolysate is performed at a pH of about 4 or greater.

77. The method of any of clauses 1 to 75, wherein the addition of the nucleophile to the hydrolysate is performed at a pH of about 6 or greater.

78. The method of any of clauses 1 to 75, wherein the addition of the nucleophile to the hydrolysate is performed at a pH of about 4 to about 8.

79. The method of any of clauses 1 to 75, wherein the addition of the nucleophile to the hydrolysate is performed at a pH of about 6 to about 8.

80. The method of any of clauses 1 to 79, wherein the alcohol is selected from the group consisting of ethanol, butanol, iso-butanol, and iso-propanol.

81. The method of any of clauses 1 to 79, wherein the alcohol is ethanol.

82. The method of any of clauses 1 to 79, wherein the alcohol is butanol.

83. The method of any of clauses 1 to 79, wherein the alcohol is iso-butanol.

84. The method of any of clauses 1 to 79, wherein the alcohol is iso-propanol.

85. The method of any of clauses 1 to 84, wherein a bio-product is formed in the hydrolysate.

86. The method of clause 85, wherein the bio-product is selected from the group consisting of a lactic acid, a succinic acid, an acrylic acid, and a 3-hydroxy propionic acid.

87. The method of clause 85, wherein the bio-product is a lactic acid.

88. The method of clause 85, wherein the bio-product is a succinic acid.

89. The method of clause 85, wherein the bio-product is an acrylic acid.

90. The method of clause 85, wherein the bio-product is a 3-hydroxy propionic acid.

91. A method of fermenting a lignocellulosic biomass, the method comprising the steps of

pretreating the lignocellulosic biomass to provide a slurry;

adding a nucleophile to the slurry to remove fermentation inhibitors from the slurry; and

adding a microorganism to the slurry to produce an alcohol,

wherein a sufficient amount of the nucleophile is added to deactivate carbonyl-containing fermentation inhibitors in the slurry.

92. The method of clause 91, wherein the slurry is not separated into a solid fraction and a liquid fraction prior to addition of the nucleophile.

93. The method of clause 91, wherein the slurry is separated into a solid fraction and a liquid fraction prior to addition of the nucleophile.

94. The method of any one of clauses 91 to 93, further comprising the step of adding one or more cellulases to the slurry resulting in hydrolysis of the slurry.

95. The method of clause 94, wherein the hydrolysis and the production of the alcohol are simultaneous.

96. The method of any one of clauses 91 to 95, wherein the pH of the slurry is adjusted prior to addition of the nucleophile.

97. The method of any one of clauses 93 to 96, wherein the liquid fraction is concentrated prior to addition of the nucleophile.

98. The method of clause 97, wherein the pH of the concentrated liquid fraction is adjusted prior to addition of the nucleophile.

99. The method of any of clauses 91 to 98, wherein the pretreatment step increases the accessibility of celluloses to cellulosic enzymes.

100. The method any of clauses 91 to 99, wherein the pretreatment step comprises adding an organic solvent or an ionic liquid.

101. The method of any of clauses 91 to 100, wherein the pretreatment step comprises pretreating the lignocellulosic biomass with saturated steam.

102. The method of any of clauses 91 to 100, wherein the pretreatment step is selected from the group consisting of chemical pretreatment, steam explosion, organosolv pretreatment, ammonia fibre explosion (AFEX), ionic liquid pretreatment, and biological pretreatment.

103. The method of any of clauses 91 to 102, wherein the pretreatment step comprises adding an acid selected from the group consisting of sulfuric acid, phosphoric acid, nitric acid, hydrochloric acid, and combinations thereof.

104. The method of any of clauses 91 to 103, wherein the pretreatment step comprises adding a base selected from the group consisting of sodium hydroxide, calcium hydroxide, potassium hydroxide, ammonia, and combinations thereof.

105. The method of any of clauses 91 to 104, wherein the pretreatment step comprises addition of an acid.

106. The method of clause 105, wherein the pretreatment step comprises adding less than 5% w/w acid.

107. The method of clause 105, wherein the pretreatment step comprises adding about 1% w/w acid.

108. The method of any of clauses 91 to 107, wherein the lignocellulosic biomass is an agricultural biomass.

109. The method of any of clauses 91 to 108, wherein the lignocellulosic biomass is selected from the group consisting of corn, corn stover, corn cobs, wood chips, softwood wood chips, hardwood wood chips, wheat straw, rice straw, hybrid poplar, sugarcane bagasse, switchgrass, miscanthus, forest thinnings, forest residues, agricultural residues, and combinations thereof.

110. The method of any of clauses 91 to 108, wherein the lignocellulosic biomass is wood.

111. The method of any of clauses 91 to 110, wherein the pretreatment step comprises organosolv pulping.

112. The method of any of clauses 91 to 111, wherein the pretreatment step comprises disrupting the lignocellulosic matrix.

113. The method of any of clauses 91 to 112, wherein the pretreatment step comprises soaking in sulfuric acid.

114. The method of any of clauses 91 to 113, wherein the pretreatment step comprises soaking in sulfuric acid at an elevated temperature.

115. The method of any of clauses 91 to 114, wherein one or more hemicellulose sugars are recovered after the pretreatment step.

116. The method of any of clauses 91 to 115, wherein the pH of the slurry is about 1.8.

117. The method of any of clauses 91 to 116, wherein the slurry comprises sugars selected from the group consisting of glucose, mannose, xylose, galactose, arabinose, and combinations thereof.

118. The method of any of clauses 91 to 117, wherein the slurry comprises sugar degradation compounds selected from the group consisting of acetic acid, levulinic acid, formic acid, furfural, hydroxymethylfurfural (HMF), vanillin, cinnamaldehyde, syringaldehyde, phenolic and aromatic compounds, and combinations thereof.

119. The method of any of clauses 91 to 118, wherein the slurry is substantially not fermentable.

120. The method of any of clauses 93 to 119, wherein the pH of the liquid fraction is about 1.8.

121. The method of any of clauses 93 to 120, wherein the liquid fraction comprises sugars selected from the group consisting of glucose, mannose, xylose, galactose, arabinose, and combinations thereof.

122. The method of any of clauses 93 to 121, wherein the liquid fraction comprises sugar degradation compounds selected from the group consisting of acetic acid, levulinic acid, formic acid, furfural, hydroxymethylfurfural (HMF), vanillin, cinnamaldehyde, syringaldehyde, phenolic and aromatic compounds, and combinations thereof.

123. The method of any of clauses 93 to 122, further comprising the step of contacting the solid fraction with hydrolytic enzymes to provide monomeric sugars.

124. The method of any of clauses 91 to 123, wherein the fermentation inhibitor is a carbonyl-containing compound.

125. The method of any of clauses 91 to 123, wherein the fermentation inhibitor is a ketone or an aldehyde.

126. The method of any of clauses 91 to 123, wherein the fermentation inhibitor is an aromatic ketone or an aromatic aldehyde.

127. The method of any of clauses 91 to 123, wherein the fermentation inhibitor is an α,β-unsaturated ketone or an α,β-unsaturated aromatic aldehyde.

128. The method of any of clauses 91 to 127, wherein the nucleophile is added to the slurry prior to addition of the microorganism.

129. The method of any of clauses 91 to 128, wherein the nucleophile is added to the slurry to provide slurry that is substantially free of the microorganism.

130. The method of any of clauses 91 to 129, wherein adding the slurry with the nucleophile takes place at about 60° C. and at a pH of about 6.0 for about 2 hours.

131. The method of any of clauses 93 to 130, wherein adding the concentrated liquid fraction with the nucleophile takes place at about 60° C. and at a pH of about 6.0 for about 2 hours.

132. The method of any of clauses 91 to 131, wherein the nucleophile is an amino acid.

133. The method any of clauses 91 to 131, wherein the nucleophile is an amino acid selected from the group consisting of glycine, alanine, valine, leucine, isoleucine, proline, phenylalanine, tyrosine, tryptophan, serine, threonine, cysteine, methionine, asparagine, glutamine, lysine, histidine, arginine, aspartate, glutamate, and combinations thereof.

134. The method of any of clauses 91 to 131, wherein the nucleophile is an amino acid selected from the group consisting of cysteine, histidine, tryptophan, asparagine, lysine, and combinations thereof.

135. The method of any of clauses 91 to 131, wherein the nucleophile comprises cysteine, histidine, or a combination thereof.

136. The method of any of clauses 91 to 131, wherein the nucleophile is cysteine, histidine, or a combination thereof.

137. The method of any of clauses 91 to 131, wherein the nucleophile consists essentially of cysteine, histidine, or a combination thereof.

138. The method of any of clauses 91 to 131, wherein the nucleophile consists of cysteine, histidine, or a combination thereof.

139. The method of any of clauses 91 to 131, wherein the nucleophile is cysteine or histidine.

140. The method of any of clauses 91 to 131, wherein the nucleophile is cysteine.

141. The method of any of clauses 91 to 131, wherein the nucleophile consists essentially of cysteine.

142. The method of any of clauses 91 to 131, wherein the nucleophile consists of cysteine.

143. The method of any of clauses 140 to 142, wherein the concentration of cysteine is about 5.0 mM.

144. The method of any of clauses 91 to 131, wherein the nucleophile is histidine.

145. The method of any of clauses 91 to 131, wherein the nucleophile consists essentially of histidine.

146. The method of any of clauses 91 to 131, wherein the nucleophile consists of histidine.

147. The method of any of clauses 91 to 131, wherein the nucleophile is glycine.

148. The method of any of clauses 91 to 131, wherein the nucleophile consists essentially of glycine.

149. The method of any of clauses 91 to 131, wherein the nucleophile consists of glycine.

150. The method of any of clauses 91 to 149, wherein the slurry is adjusted to a pH of about 6 before addition of the nucleophile.

151. The method of any of clauses 91 to 149, wherein the slurry is adjusted to a pH of about 6 before addition of the nucleophile.

152. The method of any of clauses 93 to 149, wherein the liquid fraction is adjusted to a pH of about 6 after addition of the nucleophile.

153. The method of any of clauses 93 to 149, wherein the liquid fraction is adjusted to a pH of about 6 after addition of the nucleophile.

154. The method of any of clauses 91 to 153, wherein the microorganism is a yeast.

155. The method of any of clauses 91 to 153, wherein the microorganism is Saccharomyces cerevisiae.

156. The method of any of clauses 91 to 153, wherein the microorganism is a bacteria.

157. The method of any of clauses 91 to 153, wherein the microorganism is E Coli.

158. The method of any of clauses 91 to 153, wherein the microorganism is Zymomonas mobilis.

159. The method of any of clauses 91 to 153, wherein the microorganism is Clostridium sp.

160. The method of any of clauses 91 to 153, wherein the microorganism is Clostridium acetobutylicum.

161. The method of any of clauses 91 to 160, wherein the hydrolysate is adjusted to a pH of about 6 with NaOH or H₂SO₄ and sterilized by passing 0.2 μm sterile filters.

162. The method of any of clauses 91 to 161, wherein the nucleophile prevents carbonyl compounds released during the biomass pretreatments from inhibiting biomass hydrolysates fermentation.

163. The method of any of clauses 91 to 162, wherein the nucleophile has a nucleophilicity parameter (N) of about 10 or greater.

164. The method of any of clauses 91 to 162, wherein the nucleophile has a nucleophilicity parameter (N) of about 20 or greater.

165. The method of any of clauses 91 to 164, wherein the addition of the nucleophile to the slurry is performed at a temperature of about 50° C. to about 100° C.

166. The method of any of clauses 91 to 164, wherein the addition of the nucleophile to the slurry is performed at a temperature of about 50° C. to about 90° C.

167. The method of any of clauses 91 to 164, wherein the addition of the nucleophile to the slurry is performed at a temperature of about 60° C. to about 80° C.

168. The method of any of clauses 91 to 164, wherein the addition of the nucleophile to the slurry is performed at a temperature of about 70° C. to about 80° C.

169. The method of any of clauses 91 to 168, wherein the addition of the nucleophile to the slurry is performed at a pH of about 4 or greater.

170. The method of any of clauses 91 to 168, wherein the addition of the nucleophile to the slurry is performed at a pH of about 6 or greater.

171. The method of any of clauses 91 to 168, wherein the addition of the nucleophile to the slurry is performed at a pH of about 4 to about 8.

172. The method of any of clauses 91 to 168, wherein the addition of the nucleophile to the slurry is performed at a pH of about 6 to about 8.

173. The method of any of clauses 91 to 172, wherein the alcohol is selected from the group consisting of ethanol, butanol, iso-butanol, and iso-propanol.

174. The method of any of clauses 91 to 172, wherein the alcohol is ethanol.

175. The method of any of clauses 91 to 172, wherein the alcohol is butanol.

176. The method of any of clauses 91 to 172, wherein the alcohol is iso-butanol.

177. The method of any of clauses 91 to 172, wherein the alcohol is iso-propanol.

178. The method of any of clauses 91 to 177, wherein a bio-product is formed in the slurry.

179. The method of clause 178, wherein the bio-product is selected from the group consisting of a lactic acid, a succinic acid, an acrylic acid, and a 3-hydroxy propionic acid.

180. The method of clause 178, wherein the bio-product is a lactic acid.

181. The method of clause 178, wherein the bio-product is a succinic acid.

182. The method of clause 178, wherein the bio-product is an acrylic acid.

183. The method of clause 178, wherein the bio-product is a 3-hydroxy propionic acid.

184. A method of increasing the sugar consumption rate during fermentation of a lignocellulosic biomass, the method comprising the steps of

pretreating the lignocellulosic biomass to provide a hydrolysate;

adding a nucleophile to the hydrolysate; and

adding a microorganism to the hydrolysate to produce an alcohol.

### BRIEF DESCRIPTION OF THE DRAWINGS

FIG. 1A shows the ethanol production in the fermentation of hydrolysates detoxified by various amino acids (0.2% w/v) at pH 6 and 60° C. for 2 hours.

FIG. 1B shows the sugar consumption in the fermentation of hydrolysates detoxified by various amino acids (0.2% w/v) at pH 6 and 60° C. for 2 hours.

FIG. 1C shows the HMF consumption in the fermentation of hydrolysates detoxified by various amino acids (0.2% w/v) at pH 6 and 60° C. for 2 hours.

FIG. 2A shows the effect of temperature on ethanol production from the hydrolysates detoxified by cysteine (pH 6.0 for 2 hours).

FIG. 2B shows the effect of temperature on ethanol production from the hydrolysates detoxified by glycine (pH 6.0 for 2 hours).

FIG. 3A shows the effect of pH on cysteine detoxification of hydrolysates at 60° C.

FIG. 3B shows the effect of pH on glycine detoxification of hydrolysates at 60° C.

FIG. 3C shows the effect of pH on glycine detoxification of hydrolysates at 80° C.

FIG. 4A shows the effects of vanillin and cinnamaldehyde on ethanol production following detoxification of model inhibitors with cysteine and glycine.

FIG. 4B shows the ethanol production from detoxified sugar solution with cinnamaldehyde (2.5 mM) following detoxification of model inhibitors with cysteine and glycine.

FIG. 5A shows the proposed reaction between cysteine and cinnamaldehyde.

FIG. 5B shows analysis of the detoxified products after reacting cysteine with cinnamaldehyde, with two major peaks presented at high intensities with 236.07 and 357.09 ions [M+1.].

FIG. 5C shows MS/MS analysis of the 236.07 peak. after reacting cysteine with cinnamaldehyde.

FIG. 5D shows MS/MS analysis of the 357.09 peak after reacting cysteine with cinnamaldehyde.

Various embodiments of the invention are described herein as follows. In one aspect, a method of fermenting a lignocellulosic biomass is provided. The method comprises the steps of pretreating the lignocellulosic biomass to provide a hydrolysate; adding a nucleophile to the hydrolysate; and adding a microorganism to the hydrolysate to produce an alcohol, wherein a sufficient amount of the nucleophile is added to deactivate carbonyl-containing fermentation inhibitors in the hydrolysate.

In the various aspects, methods of fermenting a lignocellulosic biomass are provided. As used herein, the term “lignocellulosic biomass” refers to plant-based or plant-derived biomass comprising carbohydrate polymers (e.g., cellulose, hemicellulose), and an aromatic polymer (e.g., lignin). Lignocellulosic biomass refers to virtually any plant-derived organic matter (woody or non-woody) available for energy on a sustainable basis. Lignocellulosic biomass can include, but is not limited to, agricultural crop wastes and residues such as corn stover, wheat straw, rice straw, sugar cane bagasse, tobacco, and the like. Lignocellulosic biomass further includes, but is not limited to, various weeds of any type, such as in the Bassicacae family (e.g., Arabidopsis), woody energy crops, wood wastes and residues such as trees (e.g., dogwood), further including fruit trees, such as fruit-bearing trees, (e.g., apple trees, orange trees, and the like), softwood forest thinnings, barky wastes, sawdust, paper and pulp industry waste streams, wood fiber, and the like. Additionally grass crops, such as various prairie grasses, including prairie cord grass, switchgrass, big bluestem, little bluestem, side oats grama, and the like, have potential to be produced large-scale as additional lignocellulosic biomass sources. For urban areas, potential lignocellulosic biomass feedstock includes yard waste (e.g., grass clippings, leaves, tree clippings, brush, etc.) and vegetable processing waste. Lignocellulosic biomass is known to be the most prevalent form of carbohydrate available in nature.

Some embodiments comprise the step of pretreating the lignocellulosic biomass to provide a hydrolysate. As used herein, “pretreating” refers to any step intended to alter native lignocellulosic biomass so it can be more efficiently and economically converted to reactive intermediate chemical compounds (e.g., sugars, organic acids, etc.) that can then be further processed to a variety of value added products. Pretreatment methods can utilize acids of varying concentrations (e.g., sulfuric acids, hydrochloric acids, organic acids, etc.) and/or other components such as ammonia, ammonium, lime, and the like. Pretreatment methods can additionally or alternatively utilize hydrothermal treatments including water, heat, steam, pressurized steam, or saturated steam. The step of preheating lignocellulosic biomass provides a hydrolysate, which can comprise fermentable sugars as well as other products. In some embodiments, the pretreatment step comprises pretreating the lignocellulosic biomass with saturated steam.

Some embodiments comprise the step of adding a nucleophile to the hydrolysate. As used herein, the term “nucleophile” refers to organic molecules that contain a reactive electronegative element. In certain aspects, adding the nucleophile to the hydrolysate provides a fermentable broth. According to the present disclosure, the nucleophile can detoxify fermentation inhibitors (including carbonyl aldehydes, carbonyl ketones and carboxylic acids) in the hydrolysate (or slurry). Furthermore, as described herein, addition of the nucleophile can detoxify the hydrolysate for subsequent fermentation (or detoxify the slurry for enzymatic hydrolysis and fermentation).

Furthermore, some embodiments comprise the step of adding a microorganism to the hydrolysate to produce an alcohol. As used herein, the term “alcohol” has its generally understood meaning in the art and refers to any molecule that includes an —OH group.

In various embodiments, a sufficient amount of the nucleophile is added to deactivate carbonyl-containing fermentation inhibitors. As used herein, a “sufficient amount of the nucleophile to deactivate carbonyl-containing fermentation inhibitors” refers to an amount of the nucleophile that, when added to a substantially not fermentable hydroxylate under the reaction conditions as described in the various embodiments provided herein, is capable of reacting with carbonyl-containing fermentation inhibitors in the hydroxylate to an extent that results in a fermentable mixture. As used herein, the term “fermentable” means capable of producing ethanol at a rate greater than 0.2 g/L/h as measured by volumetric ethanol productivity for the first 6 hours of fermentation when exposed to the fermentation conditions as described in the various embodiments provided herein.

In some embodiments, the hydrolysate is filtered into a solid fraction and a liquid fraction prior to addition of the nucleophile to the liquid fraction. The means to filter the solid fraction and the liquid fraction may be performed in any method known to a skilled artisan. In other embodiments, the liquid fraction is concentrated prior to addition of the nucleophile. The means to concentrate the liquid fraction may be performed in any method known to a skilled artisan. In yet other embodiments, the pH of the concentrated liquid fraction is adjusted prior to addition of the nucleophile. The means to adjust the pH of the liquid fraction may be performed in any method known to a skilled artisan.

In some embodiments, the pretreatment step increases the accessibility of celluloses to cellulosic enzymes. As used herein, the term “cellulosic enzyme” has its generally accepted meaning in the art and refers to an enzyme capable of reacting with cellulose.

In other embodiments, the pretreatment step comprises adding an organic solvent or an ionic liquid. As used herein, the term “organic solvent” refers to solvents which are generally non-polar, polar aprotic, or polar protic solvents. Organic solvents include, but are not limited to, tetrahydrofuran, acetonitrile, diethyl ether, methyl t-butyl ether, ethyl acetate, pentane, hexane, heptane, cyclohexane, benzene, toluene, methanol, ethanol, as well as halogenated solvents such as chloroform, dichloromethane, carbon tetrachloride, 1,2-dichloroethane, or combinations thereof.

In various embodiments, the pretreatment step is selected from the group consisting of chemical pretreatment, steam explosion, organosolv pretreatment, ammonia fibre explosion (AFEX), ionic liquid pretreatment, and biological pretreatment.

Chemical Pretreatment

Chemical pretreatment refers to a pretreatment approach in which one or more chemicals (e.g., acids, alkali, organic solvents, or ionic liquids) are added to reduce or modify the recalcitrance of lignocellulosic biomass. Acid catalysts such as sulfuric acid, phosphoric acid, nitric acid, and hydrochloric acid can achieve effective fractionation of cellulose, hemicelluloses, and lignin at low concentrations (e.g., 0.5-5%). Alkali salts such as sodium hydroxide, calcium hydroxide, and potassium hydroxide, and ammonia are of promising base catalysts to disrupt the linkage between lignin and carbohydrates as well as decreasing the degree of polymerization of cellulose. Acetyl group and uronic acid derivatives on hemicelluloses are easily removed during alkaline pretreatment. Organosolv pretreatment occurs in an organic or water-organic solvent system at temperatures ranging from 100 to 250° C., optionally with the addition of acid to facilitate solubilization of hemicelluloses. In addition, ionic liquids (ILs) may be used as a pretreatment system in which solvents (e.g., imidazonium salts possessing high polarities, low melting points, and high thermal stabilities) are used to disrupt the three dimension cellulose network.

Dilute Acid Pretreatment

Dilute acid pretreatment is a process to fractionate lignocellulosic biomass in which the pretreatment conditions may be conducted at acid charges on wood from about 0.5-5%, at temperatures between about 120-215° C., and at residence times from a few seconds to approximately one hour. Sulfuric acid may be utilized as the acid in dilute acid pretreatment. This method can effectively solubilize and recover a large fraction of the hemicelluloses (80-90%) as oligomeric and monomeric sugars in the hydrolysate phase, and at the same time disrupt the lignin structure and significantly increase the cellulose accessibility to enzymes.

Steam Explosion

Steam explosion may be conducted at high pressure and temperature (e.g., between 160-240° C.) with steam (e.g., saturated steam), with a residence time ranging from a few seconds to several minutes. This pretreatment typically solubilizes part of the hemicelluloses and somewhat modifies lignin structure, thereby increasing cellulose accessibility to enzymes. Since the acetyl group is easily released at high temperature and pressure during the pretreatment and acts as acid catalyst, this process is sometimes referred as “autohydrolysis.”

Due to low sugar yields, H₂SO₄, SO₂ or CO₂ may be added to increase sugar recovery. In these instances, the acid-catalyst steam explosion turns into another form of dilute acid pretreatment, in which a vapor phase rather than aqueous phase is used for the pretreatment.

Organosolv Pretreatment

Low boiling point alcohols such as methanol and ethanol, higher alcohols such as glycerol and ethylene glycol, and other organic solvents such as ketone, ethers, and phenols have been used in organosolv pretreatment systems, in which the operating temperature range from about 100-250° C. and at a residence time from about 30 to 60 minutes. Ethanol may be utilized due to its low cost, ease of recovery, and low inhibition on fermenting microorganisms. The use of an organic solvent can effectively solubilize lignin so that a pure lignin can be recovered as a high-value byproduct, which could be an alternative for epoxy resins and phenolic powder resins. Acid catalysts may be added to increase the release of hemicellulose sugars and the extraction of lignin.

Ammonia Fibre Explosion and Ammonia Recycle Percolation

Ammonia fibre explosion (AFEX) is typically conducted at high pressure (e.g., greater than 3 MPa), at a variety of temperatures ranging from about 60° C. to about 100° C., at a residence time from about 10 to about 60 minutes, and at a solid/ammonia ratio of about 1:1-1:2. AFEX can result in modification or partial removal of lignin as well as causing swelling of the cellulose structure enhancing digestibility to cellulases. When conducted at higher temperatures (e.g., 150-180° C.), the aqueous ammonia flows through the biomass and is then recycled, in a process known as ammonia recycle percolation (ARP). The ammonia-based pretreatments produce fewer inhibitors compared to the acid-based pretreatments and therefore detoxification may not be necessary.

Ionic Liquid Pretreatment

Ionic liquid pretreatment utilizes non-derivatizing solvents with high polarities, high thermal stabilities, and low vapor pressures to enhance digestibility of cellulose under lower temperatures. Imidazonium salts such as 1-allyl-3-methylimidazolium chloride and 1-butyl-3-methylimidazolium may be used as ionic liquids in this pretreatment process.

Biological Pretreatment

Fungi that are able to produce lignin-degrading enzymes such as lignin peroxidases, laccases, and manganese peroxidases have been used to remove lignin from lignocellulosic biomass. For example, white rot fungi such as Phlebia ochraceofulva and Phanerochaete chrysosporium can achieve significant delignification.

In various embodiments, the pretreatment step comprises adding an acid selected from the group consisting of sulfuric acid, phosphoric acid, nitric acid, hydrochloric acid, and combinations thereof. In other embodiments, the pretreatment step comprises adding a base selected from the group consisting of sodium hydroxide, calcium hydroxide, potassium hydroxide, ammonia, and combinations thereof. In certain embodiments, the pretreatment step comprises adding less than 5% w/w acid. In some embodiments, the pretreatment step comprises adding about 1% w/w acid.

In various aspects, the lignocellulosic biomass is an agricultural biomass. In some embodiments, the lignocellulosic biomass is selected from the group consisting of corn, corn stover, corn cobs, wood chips, softwood wood chips, hardwood wood chips, wheat straw, rice straw, hybrid poplar, sugarcane bagasse, switchgrass, miscanthus, forest thinnings, forest residues, agricultural residues, and combinations thereof. In some embodiments, the lignocellulosic biomass is wood.

In some embodiments, the pretreatment step comprises addition of an acid. In other aspects, the pretreatment step comprises organosolv pulping.

In various embodiments, the pretreatment step comprises disrupting the lignocellulosic matrix. As described by the present disclosure, the disruption of the lignocellulosic matrix can comprise a method utilized as the pretreatment step of the lignocellulosic biomass.

In some embodiments, the pretreatment step comprises soaking in sulfuric acid. In other embodiments, the pretreatment step comprises soaking in sulfuric acid at an elevated temperature. The means to elevate the temperature may be performed in any method known to a skilled artisan.

In certain aspects, one or more hemicellulose sugars are recovered after the pretreatment step. As used herein, the term “hemicellulose sugars” refers to sugars indicative of hemicellulose, i.e. xylose, arabinose, mannose, galactose, mannuronic acid and galacturonic acid. According to various exemplary embodiments of the present disclosure, hemicellulose sugars may be present as polymers and/or oligomers and/or monomers.

In certain other embodiments, the pH of the hydrolysate is about 1.8. In other embodiments, the pH of the liquid fraction is about 1.8. In various aspects, the hydrolysate comprises sugars selected from the group consisting of glucose, mannose, xylose, galactose, arabinose, and combinations thereof. In other aspects, the liquid fraction comprises sugars selected from the group consisting of glucose, mannose, xylose, galactose, arabinose, and combinations thereof.

In other aspects, the hydrolysate comprises sugar degradation compounds selected from the group consisting of acetic acid, levulinic acid, formic acid, furfural, hydroxymethylfurfural (HMF), vanillin, cinnamaldehyde, syringaldehyde, phenolic and aromatic compounds, and combinations thereof. As used herein, the term “sugar degradation compounds” refers to one or more compounds that are produced upon the degradation of sugar. In yet other aspects, the liquid fraction comprises sugar degradation compounds selected from the group consisting of acetic acid, levulinic acid, formic acid, furfural, hydroxymethylfurfural (HMF), vanillin, cinnamaldehyde, syringaldehyde, phenolic and aromatic compounds, and combinations thereof.

In certain aspects, the hydrolysate is substantially not fermentable. As used herein, the term “substantially not fermentable” means incapable of producing ethanol at any rate greater than 0.2 g/L/h as measured by volumetric ethanol productivity for the first 6 hours of fermentation when exposed to the fermentation conditions as described in the various embodiments provided herein. In some embodiments, the method further comprises the step of contacting the solid fraction with hydrolytic enzymes to provide monomeric sugars.

In various embodiments, the fermentation inhibitor is a carbonyl-containing compound. In other embodiments, the fermentation inhibitor is a ketone or an aldehyde. In yet other embodiments, the fermentation inhibitor is an aromatic ketone or an aromatic aldehyde. In some embodiments, the fermentation inhibitor is an α,β-unsaturated ketone or an α,β-unsaturated aromatic aldehyde.

In certain aspects, a fermentable broth is formed prior to addition of the microorganism. In some aspects, the microorganism is not added until the fermentable broth is formed according to the method of the present disclosure. In other aspects, the nucleophile is added to the hydrolysate prior to addition of the microorganism. In this regard, the microorganism is not added until the hydrolysate until after addition of the nucleophile according to the method of the present disclosure. In certain aspects, the nucleophile is added to the hydrolysate prior to addition of the microorganism. In yet other aspects, the nucleophile is added to the hydrolysate that is substantially free of the microorganism. In this regard, the hydrolysate or the fermentable broth is “substantially free” of the microorganism following addition of the nucleophile the hydrolysate. As used herein, the term “substantially free” refers to zero or nearly no detectable amount of a material, quantity, or item. For example, the amount can be less than 2 percent, less than 0.5 percent, or less than 0.1 percent of the material, quantity, or item.

In various embodiments, the step of adding the hydrolysate with the nucleophile takes place at about 60° C. and at a pH of about 6.0 for about 2 hours. In other embodiments, the step of adding the concentrated liquid fraction with the nucleophile takes place at about 60° C. and at a pH of about 6.0 for about 2 hours.

In some embodiments, the nucleophile is an amino acid. In other embodiments, the nucleophile is an amino acid selected from the group consisting of glycine, alanine, valine, leucine, isoleucine, proline, phenylalanine, tyrosine, tryptophan, serine, threonine, cysteine, methionine, asparagine, glutamine, lysine, histidine, arginine, aspartate, glutamate, and combinations thereof. In yet other embodiments, the nucleophile is an amino acid selected from the group consisting of cysteine, histidine, tryptophan, asparagine, lysine, and combinations thereof.

In some embodiments, the nucleophile comprises cysteine, histidine, or a combination thereof. In other embodiments, the nucleophile is cysteine, histidine, or a combination thereof. In yet other embodiments, the nucleophile consists essentially of cysteine, histidine, or a combination thereof. In certain embodiments, the nucleophile consists of cysteine, histidine, or a combination thereof.

In some embodiments, the nucleophile is cysteine or histidine. In other embodiments, the nucleophile is cysteine. In yet other embodiments, the nucleophile consists essentially of cysteine. In certain embodiments, the nucleophile consists of cysteine. In certain aspects, the concentration of cysteine is about 5.0 mM.

In other embodiments, the nucleophile is histidine. In yet other embodiments, the nucleophile consists essentially of histidine. In certain embodiments, the nucleophile consists of histidine.

In other embodiments, the nucleophile is glycine. In yet other embodiments, the nucleophile consists essentially of glycine. In certain embodiments, the nucleophile consists of glycine.

In some embodiments, the hydrolysate is adjusted to a pH of about 6 before addition of the nucleophile. In other embodiments, the liquid fraction is adjusted to a pH of about 6 after addition of the nucleophile. In some embodiments, the hydrolysate is adjusted to a pH of about 6 before addition of the nucleophile. In other embodiments, the liquid fraction is adjusted to a pH of about 6 after addition of the nucleophile.

In certain aspects, the microorganism is a yeast. The term “yeast” refers to a phylogenetically diverse grouping of single-celled fungi. Yeast do not form a specific taxonomic or phylogenetic grouping, but instead comprise a diverse assemblage of unicellular organisms that occur in the Ascomycotina and Basidiomycotina. Collectively, about 100 genera of yeast have been identified, comprising approximately 1,500 species (see Kurtzman and Fell, “Yeast Systematics And Phylogeny: Implications Of Molecular Identification Methods For Studies In Ecology,” In C. A. Rosa and G. Peter, eds., The Yeast Handbook. Germany: Springer-Verlag Berlin Herdelberg, 2006). Yeast reproduce principally by budding (or fission) and derive energy from fermentation, via conversion of carbohydrates to ethanol and carbon dioxide. Examples of some yeast genera include, but are not limited to: Agaricostilbum, Ambrosiozyma, Arthroascus, Arxula, Ashbya, Babjevia, Bensingtonia, Botryozyma, Brettanomyces, Bullera, Candida, Clavispora, Cryptococcus, Cystofilobasidium, Debaryomyces, Dekkera, Dipodascus, Endomyces, Endomycopsella, Erythrobasidium, Fellomyces, Filobasidium, Galactomyces, Geotrichum, Guilliermondella, Hansenula, Hanseniaspora, Kazachstania, Kloeckera, Kluyveromyces, Kockovaella, Kodamaea, Komagataella, Kondoa, Lachancea, Leucosporidium, Leucosporidiella, Lipomyces, Lodderomyces, Issatchenkia, Magnusiomyces, Mastigobasidium, Metschnikowia, Monosporella, Myxozyma, Nadsonia, Nematospora, Oosporidium, Pachysolen, Pichia, Phaffia, Pseudozyma, Reniforma, Rhodosporidium, Rhodotorula, Saccharomyces, Saccharomycodes, Saccharomycopsis, Saturnispora, Schizoblastosporion, Schizosaccharomyces, Sirobasidium, Smithiozyma, Sporobolomyces, Sporopachydermia, Starmerella, Sympodiomycopsis, Sympodiomyces, Torulaspora, Tremella, Trichosporon, Trichosporiella, Trigonopsis, Udeniomyces, Wickerhamomyces, Williopsis, Xanthophyllomyces, Yarrowia, Zygosaccharomyces, Zygotorulaspora, Zymoxenogloea, and Zygozyma. In one embodiment, the microorganism is Saccharomyces cerevisiae.

In certain aspects, the microorganism is a bacteria. In some embodiments, the microorganism is E Coli. In other embodiments, the microorganism is Zymomonas mobilis. In yet other embodiments, the microorganism is Clostridium sp. In certain aspects, the microorganism is Clostridium acetobutylicum.

In various embodiments, the hydrolysate is adjusted to a pH of about 6 with NaOH or H₂SO₄ and sterilized by passing 0.2 μm sterile filters. In other embodiments, the nucleophile prevents carbonyl compounds released during the biomass pretreatments from inhibiting biomass hydrolysates fermentation. In certain embodiments, the nucleophile has a nucleophilicity parameter (N) of about 10 or greater. In yet other embodiments, the nucleophile has a nucleophilicity parameter (N) of about 20 or greater.

In various embodiments, the addition of the nucleophile to the hydrolysate is performed at a temperature of about 50° C. to about 100° C. In other embodiments, the addition of the nucleophile to the hydrolysate is performed at a temperature of about 50° C. to about 90° C. In yet other embodiments, the addition of the nucleophile to the hydrolysate is performed at a temperature of about 60° C. to about 80° C. In other embodiments, the addition of the nucleophile to the hydrolysate is performed at a temperature of about 70° C. to about 80° C.

In various embodiments, the addition of the nucleophile to the hydrolysate is performed at a pH of about 4 or greater. In other embodiments, the addition of the nucleophile to the hydrolysate is performed at a pH of about 6 or greater. In yet other embodiments, the addition of the nucleophile to the hydrolysate is performed at a pH of about 4 to about 8. In other embodiments, the addition of the nucleophile to the hydrolysate is performed at a pH of about 6 to about 8.

In various embodiments, the alcohol is selected from the group consisting of ethanol, butanol, iso-butanol, and iso-propanol. In other embodiments, the alcohol is ethanol. As used herein, the term “ethanol” refers to a molecule of the formula CH₃CH₂OH. In yet other embodiments, the alcohol is butanol. As used herein, the term “butanol” refers to a molecule of the formula CH₃(CH₂)₂OH. In other embodiments, the alcohol is iso-butanol. As used herein, the term “iso-butanol” refers to a molecule of the formula (CH₃)₂CHCH₂OH. In yet other embodiments, the alcohol is iso-propanol. As used herein, the term “iso-propanol” refers to a molecule of the formula (CH₃)₂CHOH.

In various embodiments, a bio-product is formed in the hydrolysate. In certain aspects, the bio-product is selected from the group consisting of a lactic acid, a succinic acid, an acrylic acid, and a 3-hydroxy propionic acid. In some embodiments, the bio-product is a lactic acid. As used herein, the term “lactic acid” refers to a molecule of the formula CH₃CH(OH)CO₂H. In other embodiments, the bio-product is a succinic acid. As used herein, the term “succinic acid” refers to a molecule of the formula (CH₂)₂(CO₂H)₂. In yet other embodiments, the bio-product is an acrylic acid. As used herein, the term “acrylic acid” refers to a molecule of the formula CH₂═CHCO₂H. In other embodiments, the bio-product is a 3-hydroxy propionic acid. As used herein, the term “3-hydroxy propionic acid” refers to a molecule of the formula HO(CH₂)₂CO₂H.

In another aspect, a second method of fermenting a lignocellulosic biomass is provided. The method comprises the steps of pretreating the lignocellulosic biomass to provide a slurry; adding a nucleophile to the slurry to remove fermentation inhibitors from the slurry; and adding a microorganism to the slurry to produce an alcohol, wherein a sufficient amount of the nucleophile is added to deactivate carbonyl-containing fermentation inhibitors in the slurry. The previously described embodiments of the first method of fermenting a lignocellulosic biomass are applicable to the second method of fermenting a lignocellulosic biomass described herein.

In various embodiments of the second method of fermenting a lignocellulosic biomass, the slurry is not separated into a solid fraction and a liquid fraction prior to addition of the nucleophile. In other embodiments, the slurry is separated into a solid fraction and a liquid fraction prior to addition of the nucleophile.

In certain aspects of the second method of fermenting a lignocellulosic biomass, the method further comprises the step of adding one or more cellulases to the slurry resulting in hydrolysis of the slurry. In some embodiments, the hydrolysis and the production of the alcohol are simultaneous, a process which may be referred to as a simultaneous saccharification and fermentation (SSF) process.

In some embodiments of the second method of fermenting a lignocellulosic biomass, the pH of the slurry is adjusted prior to addition of the nucleophile. In other embodiments, the liquid fraction is concentrated prior to addition of the nucleophile. In yet other embodiments, the pH of the concentrated liquid fraction is adjusted prior to addition of the nucleophile. In certain aspects, the addition of the nucleophile is to the liquid fraction.

In another aspect, a method of increasing the sugar consumption rate during fermentation of a lignocellulosic biomass is provided. This method comprises the steps of pretreating the lignocellulosic biomass to provide a hydrolysate; adding a nucleophile to the hydrolysate; and adding a microorganism to the hydrolysate to produce an alcohol. The previously described embodiments of the methods of fermenting a lignocellulosic biomass are applicable to the method of increasing the sugar consumption rate during fermentation of a lignocellulosic biomass described herein.

While the invention is susceptible to various modifications and alternative forms, specific embodiments are herein described in detail. It should be understood, however, that there is no intent to limit the invention to the particular forms described, but on the contrary, the intention is to cover all modifications, equivalents, and alternatives falling within the scope of the invention.

### EXAMPLE 1

### Treatment of Lignocellulosic Biomass Hydrolysates with Various Amino Acid Nucleophiles

In the instant example, various amino acid nucleophiles were analyzed to evaluate their potential for treatment of lignocellulosic biomass. For the instant example, such treatment with amino acid nucleophiles may be referred to as “detoxification,” a term that is known to the skilled artisan.

A. Pretreatment of Lignocellulosic Biomass

Loblolly pine (Pinus taeda) wood chips (1.0×1.0 cm, L×W) were collected by Forest Products Laboratory at Auburn University, located in Auburn, Ala. Dilute acid pretreatment was used to produce biomass hydrolysates in a 4.0 L Parr batch reactor (Parr Instrument Co., Moline, Ill.) as previously described (Lai, C. H., Tu, M. B., Li, M., Yu, S. Y. 2014. Remarkable solvent and extractable lignin effects on enzymatic digestibility of organosolv pretreated hardwood. Bioresource Technology, 156, 92-98). Briefly, wood chips (400 grams, dry weight) were soaked in 1% (w/w) sulfuric acid overnight (7:1 liquor/solid ratio) prior to the pretreatment, and then loaded into a reactor and treated at 170° C. for 60 minutes. After pretreatment, the pretreated slurry was separated into a solid fraction and a liquid fraction by filtration, and the biomass hydrolysates (liquid fraction) was collected for further study and the initial pH was 1.8.

To achieve higher ethanol titer in fermentation, loblolly pine hydrolysates were concentrated to approximately one-third of its original volume using a rotary evaporator (IKA RV10 basic) at 40° C. and 60 rotations per minute (rpm). The hydrolysates were first adjusted to pH 4.0 before evaporation. After the concentration, the sugar contents of the hydrolysates were analyzed by HPLC. The hydrolysates contained 55.1 g/L of total sugars with 16.8 g/L glucose, 38.3 g/L mannose, 29.2 g/L xylose and 6.0 g/L arabinose. With regard to the sugar degradation compounds, the concentrations of formic acid, acetic acid, levulinic acid and HMF were 2.1, 5.5, 0.4 and 3.7 g/L respectively. Furfural was completely removed during the evaporation. The concentrated hydrolysates (called hydrolysates hereafter) were not fermentable and used for all the detoxification and fermentation processes in this study.

B. Chemicals and Amino Acids

Glucose, mannose, galactose, xylose, and arabinose were obtained from Fluka (Milwaukee, Wis.) and Alfa Aesar (Ward Hill, Mass.). Glycine, alanine, valine, leucine, isoleucine, proline, phenylalanine, tyrosine, tryptophan, serine, threonine, cysteine, methionine, asparagine, glutamine, lysine, histidine, arginine, aspartate, and glutamate were purchased from Alfa Aesar, Sigma-Aldrich (St. Louis, Mo.) and Acros Organics (Morris Plains, N.J.). Acetic acid, levulinic acid, formic acid, furfural, hydroxymethylfurfural (HMF), vanillin, and cinnamaldehyde were purchased from Alfa Aesar, Fisher Scientific (Fair Lawn, N.J.), Aldrich (Milwaukee, Wis.) and Pickering laboratories (Mountain View, Calif.). All chemical reagents were of chromatographic grade. Stock solutions (1.0 M) of furfural, HMF, vanillin and cinnamaldehyde were prepared in ethanol (Sigma-Aldrich) separately before further use. Stock solutions (1.0 M) of acetic acid, levulinic acid and formic acid were prepared in nanopure water (Barnstead) separately. All stocks were protected from light and kept at 4° C. The stocks were used within 1 month.

C. Detoxification of Biomass Hydrolysates with Amino Acids

The biomass hydrolysates (100 mL) were treated with the 20 amino acids (0.2% w/v) respectively at 60° C. and pH 6.0 for 2 hours. The detoxification experiments were performed in a 250 mL flask and placed in a temperature-controlled water batch.

D. Microbial Fermentation

Baker yeast (Fleischmann's), S. cerevisiae, was used for all the fermentation experiments in this study. The yeast was maintained on YDP medium containing (g/L): 20 glucose, 20 peptone, 10 yeast extract and 20 agar. Isolated colony was grown in YDP liquid medium overnight and harvested as fermentation inoculum. The yeast concentration was measured using an UV-vis spectrophotometer. Inoculum of 2.0 g/L was used for all fermentation experiments.

Batch fermentation was carried out in 125 mL serum bottles containing 50 mL broths (untreated or detoxified hydrolysates, untreated or detoxified cinnamaldehyde sugar solution) without any additional nutrient supplement. All the fermentation broths were adjusted to pH 6 with NaOH or H₂SO₄ and sterilized by passing 0.2 μm sterile filters. After inoculation, the serum bottle was sealed with rubber stopper and aluminum seal, and equipped with cannulas for CO₂ release. All fermentation experiments were incubated at 30° C., spun at 150 rpm in a shaker (E24, New Brunswick Scientific). Aliquots of samples were withdrawn at 0, 1, 3, 6, 9, 12, 24, 36 and 48 hours for the time course analysis of both starting material and products. Fermentation was carried out in duplicate. The volumetric ethanol productivity and initial consumption rate of glucose (R_{G}) and mannose (R_{M}) was calculated based on the sugar consumed in the first 6 h of fermentation as described previously (Cao, D. X., Tu, M. B., Xie, R., Li, J., Wu, Y. N., Adhikari, S. 2014. Inhibitory Activity of Carbonyl Compounds on Alcoholic Fermentation by Saccharomyces cerevisiae. Journal of Agricultural and Food Chemistry, 62(4), 918-926).

E. HPLC and LC/MS Analysis

The biomass sugars, including glucose, mannose, galactose, xylose and arabinose, were quantified by integrating the peak area of the compound eluted from a HPLC system equipped with a strong cation-exchange column (Aminex HPX-87P, 300×7.8 mm), a refractive index detector (RID-10A), with column temperature of 85° C., and nanopure water as the mobile phase at a flow rate of 0.6 ml/min for a 35 min isocratic run. Ethanol, acetic acid, levulinic acid, formic acid, furfural and HMF were analyzed using an Aminex HPX-87H column (300×7.8 mm) with a refractive index detector. The elution conditions were column temperature of 45° C. and flow rate of 0.6 ml/min with 5.0 mM H₂SO₄ as the mobile phase in a 60 min isocratic run. The detoxification products were analyzed by an Ultra Performance LC system (ACQUITY UPLC, Waters) coupled with a quadrupole time-of-flight (Q-TOF) mass spectrometer with electrospray ionization (ESI) in positive ion mode, with or without C18 column chromatography operated by the Masslynx software (V4.1).

In a loop injection without a column, each sample, in H₂O, was injected directly into ion source and acquired spectrum. With column, each sample was injected onto a C18 column (ACQUITY UPLC® BEH C18, 1.7 μm, 2.1×50 mm, Waters) with a 150 μL/min flow rate of mobile phase of solution A (95% H₂O, 5% acetonitrile, 0.1% formic acid) and solution B (95% acetonitrile, 5% H₂O, 0.1% formic acid) in a 10 min gradient starting at 95% A to 5% A in 6 min and back to 95% in 8 minutes. The ion source voltages were set at 3 KV, sampling cone at 37 V and the extraction cone at 3 V. In both modes the source and desolvation temperature were maintained at 120° C. and 225° C., respectively, with the desolvation gas flow at 200 L/h. The TOF MS scan was from 200 to 800 m/z at 1 s with 0.1 s inter-scan delay using extended dynamic range acquisition with centriod data format. For real time mass calibration, direct infusion of sodium formate solution (10% formic acid/0.1M NaOH/isopropanol at a ratio of 1:1:8) at 1 sec/10 sec to ion source at 1 μL/min was used.

The instrument was calibrated at the time of data acquisition in addition to real time calibration by the lockmass. Mass accuracy at 5 ppm or less was the key for assuring the presence of target molecules. Ion source parameters such as the source temperature (gas and sample cone), mobile phase flow rate, and cone voltage were fixed throughout the study. Ions of interest were analyzed for mass accuracy, elemental composition (using accurate mass measurement of less than 5 ppm error) and isotope modeling to identify the formula. Quantification of unknowns was performed by computing intensity of the chromatogram using either the ion count in the spectrum or the peak area displayed target ion mass in the chromatogram, referencing to known amount of standard peptides acquired under the same conditions in the same time period.

MS/MS of product ion: Ions of interest, or the parent ion, was acquired M+1 or M−1 of its accurate mass, and entered as the Set Mass for the MS/MS fragmentation. Collision energy, the kinetic energy of argon gas, was adjusted so the daughter ions produced have similar intensity, with mass range up, to the parent ion.

Carbonyl compounds released during the biomass pretreatments significantly inhibition biomass hydrolysates fermentation. Without being bound by any theory, it is believed that strong nucleophilic amino acids (e.g., lysine, cysteine, and histidine) can react with carbonyl aldehydes and ketones through nucleophilic addition and detoxify the biomass hydrolysates. To examine this hypothesis, the biomass hydrolysates were detoxified with 20 amino acids (Table 1) respectively at 60° C. and pH 6 for 2 hours initially and determined the glucose and mannose consumption rate, ethanol productivity, final concentration and ethanol yield.

As demonstrated herein, five amino acids remarkably improved the fermentability of biomass hydrolysates (see Table 1). Detoxification with cysteine and histidine produced 23.14 and 23.07 g/L ethanol, respectively, at 48 hours, and detoxification with tryptophan, asparagine, and lysine generated 11.59, 11.18 and 14.40 g/L ethanol, respectively, at 48 hours, as comparing to 1.16 g/L ethanol from the untreated biomass hydrolysates.

TABLE 1
Fermentability of biomass hydrolysates detoxified by 20 amino acids (pH 6.0, 60° C. and 2 hours)

| | R_{G} ^{a} | R_{M} ^{b} | Q_{EtOH} ^{c} | C_{EtOH} ^{d} | Y_{EtOH} ^{e} | C_{HMF} ^{f} |
|---|---|---|---|---|---|---|
| Treatment | (g/L/h) | (g/L/h) | (g/L/h) | (g/L) | (g/g) | (%) |
| Glucose control | 2.10 ± 0.01 | 1.51 ± 0.03 | 1.39 ± 0.00 | 22.68 ± 0.12 | 0.43 ± 0.00 | NA |
| Untreated | 0.11 ± 0.01 | 0.34 ± 0.07 | 0.18 ± 0.00 | 1.16 ± 0.01 | 0.02 ± 0.00 | 6.37 ± 0.67 |
| Glycine | 0.39 ± 0.01 | 0.50 ± 0.05 | 0.42 ± 0.01 | 8.58 ± 0.27 | 0.16 ± 0.01 | 11.19 ± 0.20 |
| Alanine | 0.21 ± 0.02 | 0.44 ± 0.07 | 0.28 ± 0.00 | 2.14 ± 0.01 | 0.04 ± 0.00 | 11.03 ± 1.64 |
| Valine | 0.25 ± 0.00 | 0.49 ± 0.03 | 0.32 ± 0.00 | 2.69 ± 0.18 | 0.05 ± 0.00 | 12.66 ± 1.50 |
| Leucine | 0.27 ± 0.04 | 0.64 ± 0.12 | 0.34 ± 0.01 | 2.83 ± 0.19 | 0.05 ± 0.00 | 12.84 ± 1.32 |
| Isoleucine | 0.37 ± 0.02 | 0.66 ± 0.06 | 0.44 ± 0.08 | 5.03 ± 1.46 | 0.09 ± 0.03 | 17.36 ± 3.50 |
| Proline | 0.27 ± 0.05 | 0.67 ± 0.18 | 0.33 ± 0.00 | 3.12 ± 0.13 | 0.06 ± 0.00 | 13.35 ± 1.05 |
| Phenylalanine | 0.24 ± 0.01 | 0.56 ± 0.01 | 0.28 ± 0.00 | 1.87 ± 0.02 | 0.03 ± 0.00 | 12.69 ± 1.12 |
| Tyrosine | 0.25 ± 0.02 | 0.59 ± 0.05 | 0.30 ± 0.02 | 2.16 ± 0.25 | 0.04 ± 0.00 | 12.17 ± 0.92 |
| Tryptophan | 0.69 ± 0.00 | 0.89 ± 0.04 | 0.63 ± 0.01 | 11.59 ± 0.25 | 0.21 ± 0.00 | 42.12 ± 0.81 |
| Serine | 0.38 ± 0.00 | 0.65 ± 0.13 | 0.45 ± 0.00 | 3.87 ± 0.08 | 0.07 ± 0.00 | 15.66 ± 0.11 |
| Threonine | 0.36 ± 0.02 | 0.67 ± 0.03 | 0.41 ± 0.01 | 3.96 ± 0.13 | 0.07 ± 0.00 | 16.19 ± 0.87 |
| Cysteine | 2.10 ± 0.04 | 2.16 ± 0.12 | 1.77 ± 0.03 | 23.14 ± 0.10 | 0.42 ± 0.00 | 96.40 ± 0.03 |
| Methionine | 0.27 ± 0.01 | 0.63 ± 0.02 | 0.31 ± 0.00 | 3.11 ± 0.02 | 0.06 ± 0.00 | 12.78 ± 0.10 |
| Asparagine | 0.65 ± 0.01 | 0.87 ± 0.10 | 0.58 ± 0.01 | 11.18 ± 0.15 | 0.20 ± 0.01 | 39.20 ± 0.50 |
| Glutamine | 0.38 ± 0.02 | 0.73 ± 0.01 | 0.40 ± 0.00 | 3.52 ± 0.06 | 0.06 ± 0.01 | 13.38 ± 0.99 |
| Lysine | 0.80 ± 0.06 | 1.09 ± 0.13 | 0.72 ± 0.01 | 14.40 ± 0.57 | 0.26 ± 0.01 | 50.20 ± 4.97 |
| Histidine | 0.94 ± 0.00 | 1.02 ± 0.01 | 0.78 ± 0.00 | 23.07 ± 0.35 | 0.42 ± 0.01 | 81.94 ± 1.57 |
| Arginine | 0.29 ± 0.02 | 0.59 ± 0.07 | 0.34 ± 0.01 | 2.62 ± 0.03 | 0.05 ± 0.00 | 12.69 ± 0.27 |
| Aspartate | 0.43 ± 0.02 | 0.90 ± 0.01 | 0.47 ± 0.00 | 3.86 ± 0.18 | 0.07 ± 0.00 | 16.27 ± 0.30 |
| Glutamate | 0.34 ± 0.02 | 0.56 ± 0.03 | 0.41 ± 0.01 | 3.63 ± 0.23 | 0.07 ± 0.00 | 15.40 ± 0.72 |

^{a} R_{G}, glucose consumption rate in the first 6 h.
^{b} R_{M}, mannose consumption rate in the first 6 h.
^{c} Q_{EtOH}, volumetric ethanol productivity in the first 6 h.
^{d} C_{EtOH}, ethanol final concentration at 48 h.
^{e} Y_{EtOH}, ethanol yield from total glucose and mannose at 48 h.
^{f} C_{HMF}, percentage of HMF consumed at 48 h.

Similarly, detoxification with these five amino acids also improved the glucose and mannose consumption rate significantly (see Table 1). Tryptophan, cysteine, asparagine, lysine, and histidine increased the glucose consumption rate from 0.11 g/L/h (untreated) to 0.69, 2.10, 0.65, 0.80 and 0.94 g/L/h, respectively, and increased the mannose consumption rate from 0.34 g/L/h (untreated) to 0.89, 2.16, 0.87, 1.09 and 1.02 g/L/h respectively. Compared to the positive sugar control, cysteine detoxification enabled faster sugar consumption rate, especially for mannose, which was improved by 43%. Consequently, this resulted in a substantial improvement of volumetric ethanol productivity from 0.18 to 1.77 g/L/h for the detoxified hydrolysates with cysteine, the ethanol productivity was even 27% higher than that (1.39 g/L/h) from positive sugar control. This indicates that cysteine was the most effective amino acid in the detoxification process.

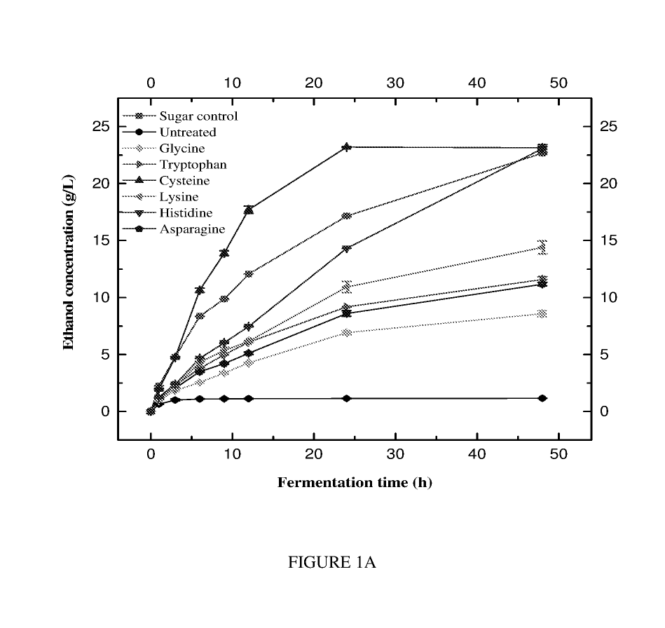

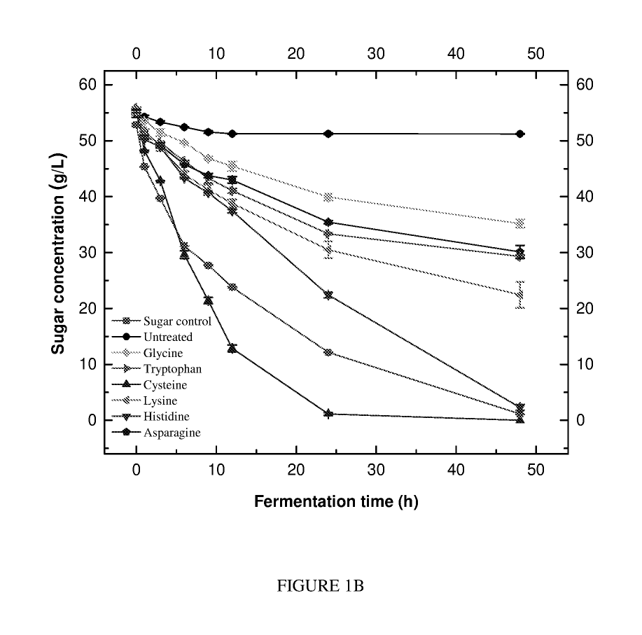

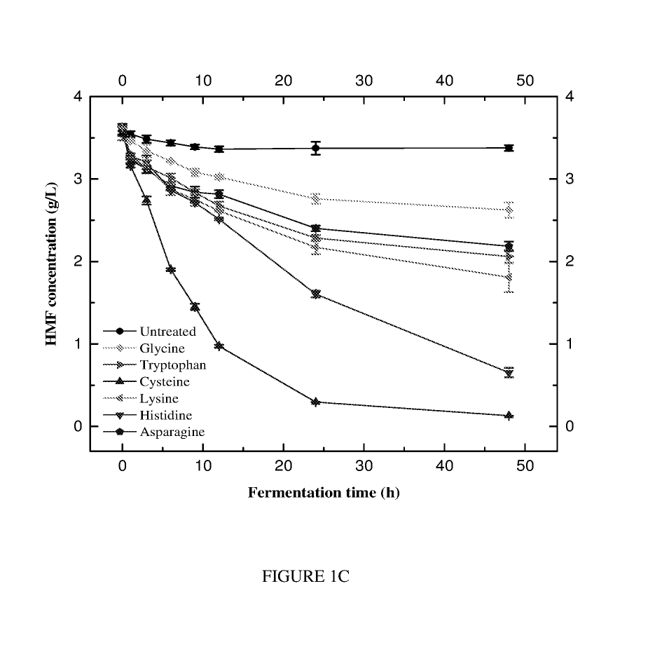

This observation was further confirmed by the highest final ethanol concentration (23.14 g/L) and ethanol yield (0.42 g/g) after 48 hours fermentation (see FIG. 1A). The second most effect amino acid was histidine, which enabled the same final ethanol yield. However, the sugar consumption rate of histidine (0.94 and 1.02 g/L/h for glucose and mannose) was 50% less than those from cysteine detoxification (see Table 1 and FIG. 1B). In addition, the same five amino acids (tryptophan, cysteine, asparagine, lysine, and histidine) detoxification resulted in a significant increase (39.20-96.40%) of HMF consumption and also cysteine was the most effective one (see FIG. 1C).

The remaining 15 amino acids showed minor improvement on ethanol yield (<0.10 g/g) in biomass hydrolysates fermentation, with the exception of glycine, which resulted in higher ethanol yield at 0.16 g/g. Based on the increase of ethanol yield, the detoxification-effective amino acids may be divided into three groups. The most effective amino acids are cysteine and histidine with final ethanol yield at 0.42 g/g. The second most effective amino acids are tryptophan, asparagine, and lysine with final ethanol yield around 0.20-0.26 g/g. The third group included glycine.

Interestingly, it was observed that the five most effective amino acids contain important functional side chains: the thiol group of cysteine, the imidazolyl group of histidine, the ε-amino group of lysine, the indolyl group of tryptophan, and the carboxamide group of arginine. The amino acid detoxification efficiency may depend on the nucleophilic reactivity of these amino acid side chains. The thiol group of cysteine has been reported as the most potent nucleophile. It has been shown that cysteine has a much higher nucleophilicity parameter (N=23.43) than all other amino acids when comparing the nucleophilicities of 16 amino acids based on their reaction with electrophilic benzhydrylium terafluoroborates. The high nucleophilicity of cysteine can be attributed to its thiol group, which exceeded the reactivities of the primary amine groups by a factor of 10⁴. Similar results have been observed when nucleophilic reactivity of amine and thiol groups were assessed by reacting with α, β-unsaturated compounds individually, in which the thiol group was around 280 times more reactive than amine group. Thus, cysteine completely detoxifies hydrolysates, resulting in higher fermentation rate and yield than all the other amino acids. The thiol group side chain in cysteine may play a critical role in detoxifying reactive carbonyl compounds in the hydrolysates. Furthermore, histidine also exhibited promising detoxification efficiency with the same ethanol yield as the cysteine detoxification. The secondary amine in the imidazole side chain of histidine makes it one of the strongest bases at neutral pH due to the low pKₐ (6.1). Histidine-containing dipeptides detoxified aldehyde compounds in biological cells as aldehyde scavengers. Therefore, the favorable detoxification effect of histidine can be attributed to its high nucleophilicity of imidazolyl group. Indeed, the side chain of cysteine, histidine and lysine often serves as important biological nucleophilic sites that are attacked by reactive aldehydes or other electrophilic toxins, forming a complex of stable products. The present disclosure indicates that cysteine, histidine, and lysine are candidates for biomass hydrolysates detoxification because they contain a secondary nucleophilic functional group, apart from the primary amine group.

Tryptophan with an indole group has been shown to react readily with phenolic aldehydes in biological systems. Asparagine with a carboxamide group can react with carbonyl compounds to form acrylamide in the maillard reaction. The side chain functional groups may play a more important role than the primary amine groups in detoxification, because a correlation between pKₐ of the primary amine group and their detoxification activity was not observed (r²<0.01). Similarly, when comparing the nucleophilicity parameters of the primary amine groups in amino acids, this difference may not be significant.

Although cysteine and histidine successfully detoxified the hydrolysates, they did not react and remove sugar degradation compounds in the detoxification process. The concentration of acetic acid (5.5 g/L), formic acid (2.1 g/L), levulinic acid (0.4 g/L), and HMF (3.7 g/L) was not changed substantially (<10%) after detoxification. This indicated that degradation compounds probably were not the major inhibitors in the biomass hydrolysates. Similar observations have been shown about the effects of sugar degradation compounds on ethanol fermentation, such as acetic acid (9 g/L) at pH 5 can increase the final ethanol yield in S. cerevisiae fermentation by 16%. It has also been shown that addition of 25 mM (3.16 g/L) HMF did not affect the ethanol production by S. cerevisiae. Therefore, nucleophilic reactions with unknown highly reactive lignin-derived carbonyl inhibitors could be the main reason for the detoxification. Although amino acids did not remove HMF from the hydrolysates during the detoxification, HMF consumption was increased significantly in the fermentation of the detoxified hydrolysates (see Table 1). HMF consumption was 6.37% in the untreated hydrolysate, but it increased from 11.9, 39.2, 42.12, 50.20, and 81.94 to 96.40% in the glycine, asparagine, tryptophan, lysine, histidine, and cysteine detoxified hydrolysates, respectively (see FIG. 1C). Overall, the increase in HMF consumption followed the same pattern as the ethanol yield (see Table 1). Thus, yeast cells were able to convert furans to their corresponding alcohols as the major products and acids as the minor products using multiple enzymes such as alcohol dehydrogenase and aldehyde dehydrogenase.

In summary, five amino acids (cysteine, histidine, lysine, tryptophan, and arginine) with important side chain functional groups showed very good detoxification efficiency and cysteine was one of the most effective one.

### EXAMPLE 2

### Effects of Temperature on the Nucleophilic Treatment Process with Cysteine and Glycine

In the instant example, the effect of temperature on treatment of lignocellulosic biomass using the nucleophiles cysteine and glycine was analyzed. For the instant example, such treatment with cysteine and glycine may be referred to as “detoxification,” a term that is known to the skilled artisan.

Preparation of the lignocellulosic biomass and the treatment thereof was performed as described in Example 1. To investigate the effect of temperature on detoxification efficiency, the biomass hydrolysates were treated at pH 6.0 with cysteine (0.2% w/v) under various temperatures (20, 40, 60, and 80° C.).

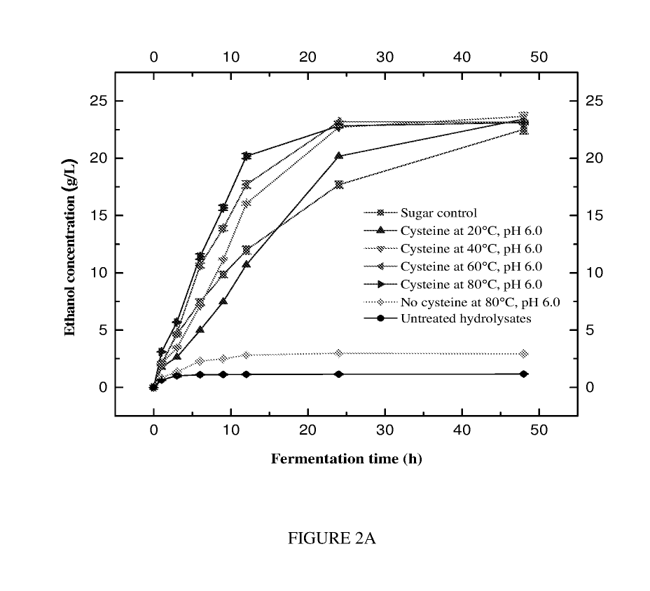

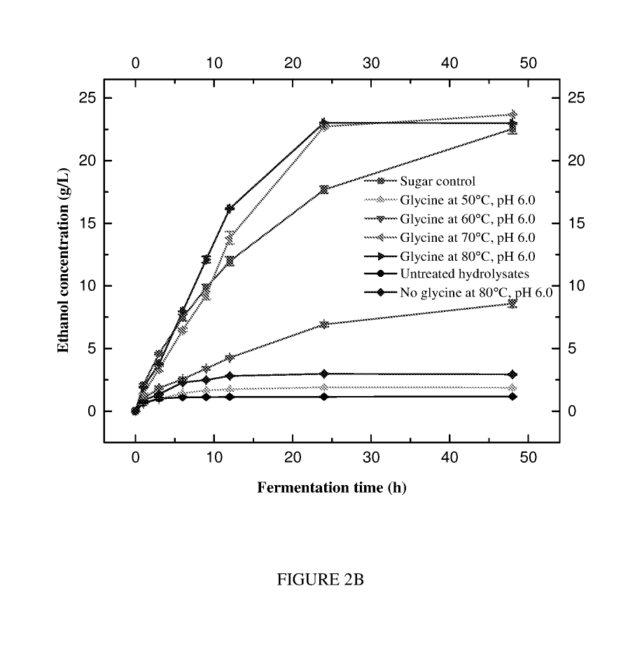

As shown in FIG. 2A and FIG. 2B, higher temperatures resulted in higher fermentation rate in cysteine and glycine detoxified hydrolysates fermentation. For cysteine detoxification, the volumetric ethanol productivity increased from by 43-130% from 0.83 to 1.19, 1.77, and 1.90 g/L/h respectively when temperature was changed from 20 to 40, 60, and 80° C. (see FIG. 2A). However, the final ethanol concentrations of the samples were the same (about 23.34 g/L) and the final ethanol yields were approximately the same (0.42 g/g) as well. The undetoxified hydrolysates was also treated at 80° C. and resulted in negligible fermentation. This indicated that cysteine is an excellent detoxification reagent and the detoxification process in temperature-dependent.

For glycine detoxification, the temperature demonstrated a dramatic improvement on the detoxified hydrolysates fermentation (see FIG. 2B). At 50 and 60° C., the ethanol productivity only improved slightly from 0.18 (untreated) to 0.24 and 0.42 g/L/h, respectively. The final ethanol concentrations were 1.87 and 8.58 g/L, respectively. However, at 70 and 80° C., the ethanol productivity was increased significantly to 1.08 and 1.33 g/L/h, and the final ethanol concentrations reached 23.00 g/L, which were comparable the cysteine detoxification. This result suggests that glycine with side chain functional group can also detoxify the hydrolysates, and the primary amine group in amino acids can react with carbonyl groups at relatively high temperature (80° C.). In conclusion, both cysteine and glycine detoxification were temperature dependent.

### EXAMPLE 3

### Effects of pH on the Nucleophilic Treatment Process with Cysteine and Glycine

In the instant example, the effect of pH on treatment of lignocellulosic biomass using the nucleophiles cysteine and glycine was analyzed. For the instant example, such treatment with cysteine and glycine may be referred to as “detoxification,” a term that is known to the skilled artisan.

Preparation of the lignocellulosic biomass and the treatment thereof was performed as described in Example 1. To investigate the effect of pH on detoxification process, the biomass hydrolysates were treated with cysteine at 60° C. and various pHs (2.0, 4.0, and 6.0).

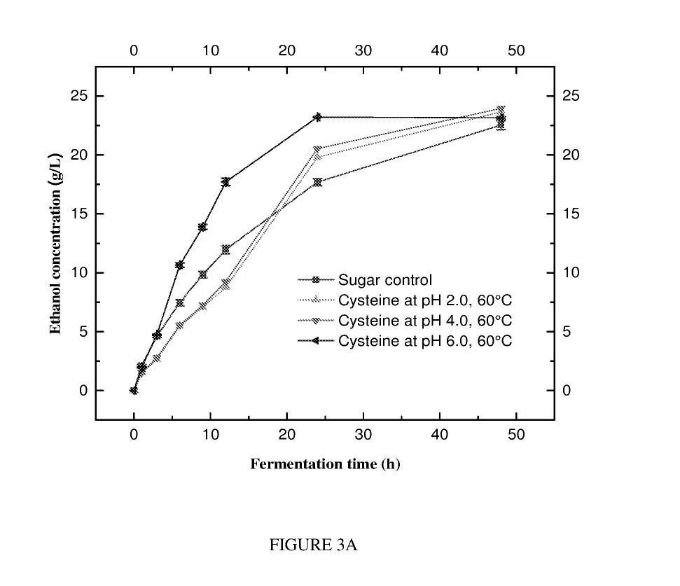

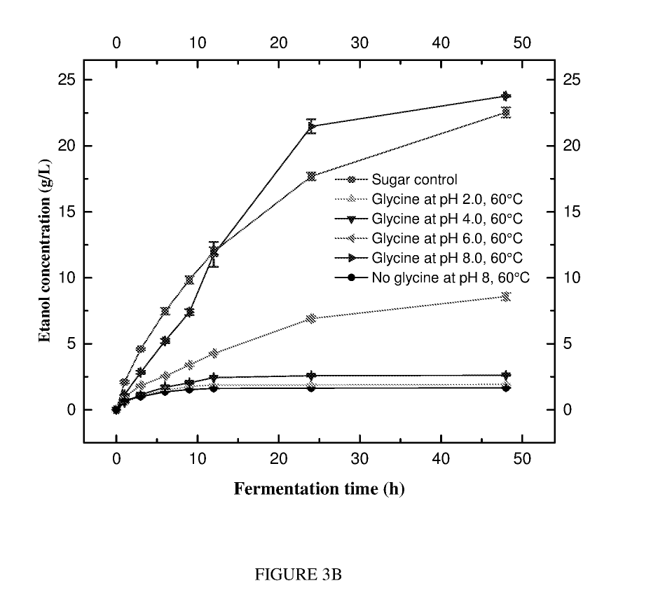

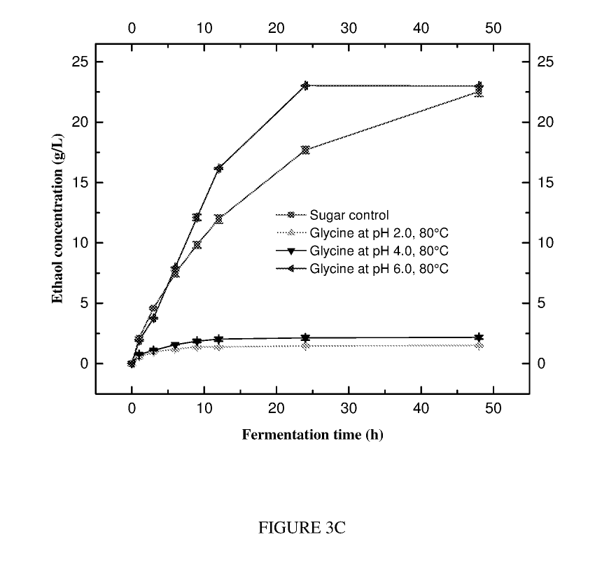

To examine whether pH affects the detoxification process, the biomass hydrolysates were detoxified with cysteine and glycine for 2 hours at various pH (2.0, 4.0, 6.0, and 8.0) while maintaining the temperature at 60° C. (see FIGS. 3A, 3B, and 3C). The results showed that the increase of pH in detoxification process enhanced the fermentation rate and yield (in glycine detoxification) considerably. For cysteine detoxification, the resulting volumetric ethanol productivity increased from by 92% from 0.92 to 1.77 g/L/h when pH was increased from 2 and 4 to 6 (see FIG. 3A). However, there was no change observed for ethanol productivity when the pH was increased from 2.0 to 4.0. Under these three pH condition, it was observed that the final ethanol concentration was the same (23.56 g/L). This indicated that cysteine detoxification was effective between pH 2.0-6.0 and higher pH facilitated more effective detoxification.

For glycine detoxification, higher pH in detoxification not only improved the fermentation rate significantly but also enhanced the final ethanol concentration substantially (see FIG. 3B). At pH 2.0 and 4.0, the detoxified biomass hydrolysates were not fermentable. At pH 6.0 and 8.0, the resulting ethanol productivity increased from 0.18 (untreated) to 0.42 and 0.87 g/L/h, respectively. The final ethanol concentrations reached 8.58 and 23.77 g/L, respectively. Meanwhile, the undetoxified biomass hydrolysates (incubated at pH 8.0) were not fermentable. This indicated that glycine detoxification required higher pH conditions (>6.0) and the detoxification process was strongly pH-dependent.

To further examine whether this pH-dependence could be overcome by higher temperature, the biomass hydrolysates were detoxified at pH 2.0, 4.0, and 6.0 under 80° C. (see FIG. 3C). The results showed the detoxified hydrolysates at pH 2.0 and 4.0 were still not fermentable, although higher temperature improved the fermentation for the hydrolysates detoxified at pH 6.0. The temperature and pH dependence of detoxification was likely related to the chemical reaction between amino acids and carbonyl inhibitors (aldehydes) in the hydrolysates. Amino acids reaction with aldehydes to form Schiff base has been shown to be temperature dependent and the reaction was favored in high temperature. Compared with glycine, cysteine contains another side chain functional group-thiol group, which can react readily with carbonyl compounds. Consequently, it can react faster with carbonyl inhibitors even at middle conditions. In conclusion, both cysteine and glycine detoxification were pH dependent.

### EXAMPLE 4

### Detoxification of Model Inhibition Compound with Cysteine and Glycine and Identification of Potential Detoxification Products

For the instant example, treatment with cysteine and glycine may be referred to as “detoxification,” a term that is known to the skilled artisan.

Preparation of the lignocellulosic biomass and the treatment thereof was performed as described in Example 1. After detoxification, the detoxified hydrolysates were cooled to room temperature and then readjusted to pH 6.0 if needed. Similarly, for the model compound (cinnamaldehyde) detoxification, glucose (˜20 g/L) was mixed with 2.5 mM cinnamaldehyde, and then treated with 5 mM cysteine or glycine at 60° C. for 2 hours prior to fermentation. A mixture of glucose (17.7 g/L) and mannose (35.1 g/L) without inhibitors was used as a positive sugar control for the fermentation

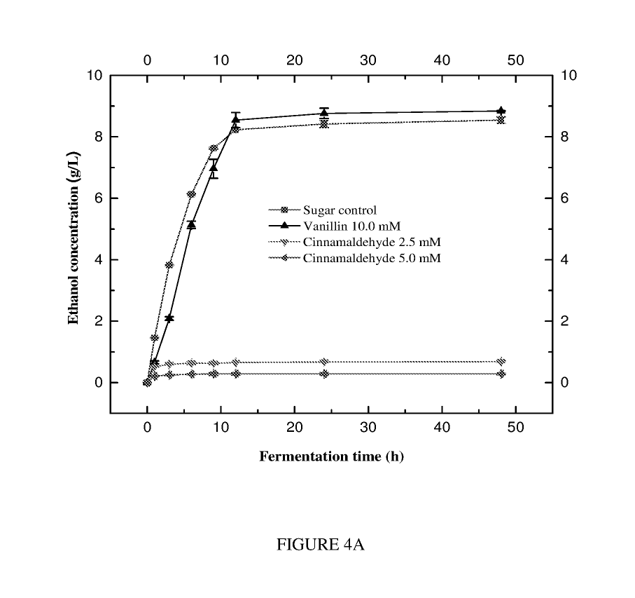

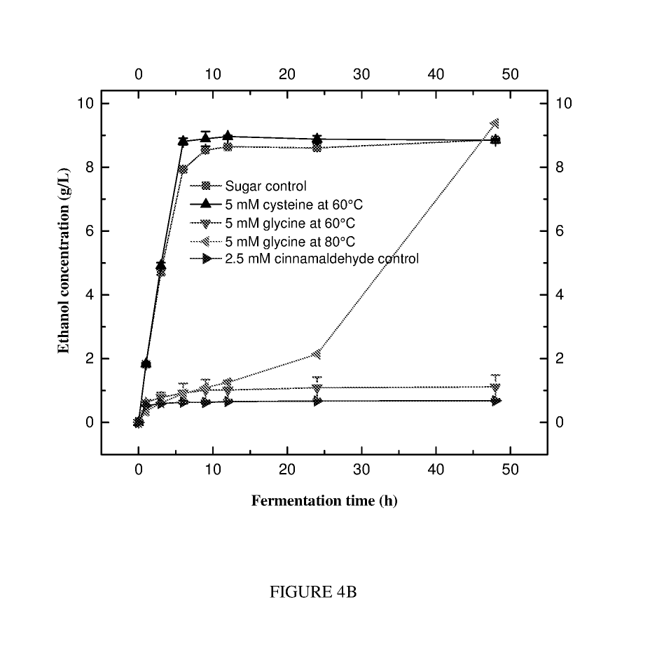

To ascertain the potential reaction mechanism in amino acids detoxification of biomass hydrolysates, cinnamaldehyde was detoxified in glucose solution with cysteine and glycine. Cinnamaldehyde was chosen as a model inhibition compound, because it has been identified in wood hydrolysates and had strong inhibition on alcoholic fermentation. Although vanillin has been evaluated as a model inhibitor, preliminary results showed 2.5 mM cinnamaldehyde inhibited glucose fermentation significantly while 10 mM vanillin did not inhibit the final ethanol concentration and yield (see FIG. 4A).

Comparing the detoxification efficiency of cysteine and glycine on cinnamaldehyde (see FIG. 4B), it was observed that 5.0 mM cysteine detoxified cinnamaldehyde very effectively and enabled a faster fermentation comparable to the glucose control. Glycine did not detoxify the cinnamaldehyde at 60° C. and the glucose solution became fermentable after it was detoxified at 80° C. This confirmed previous observations on detoxification of biomass hydrolysates that cysteine has higher detoxification activity towards biomass hydrolysates than glycine.

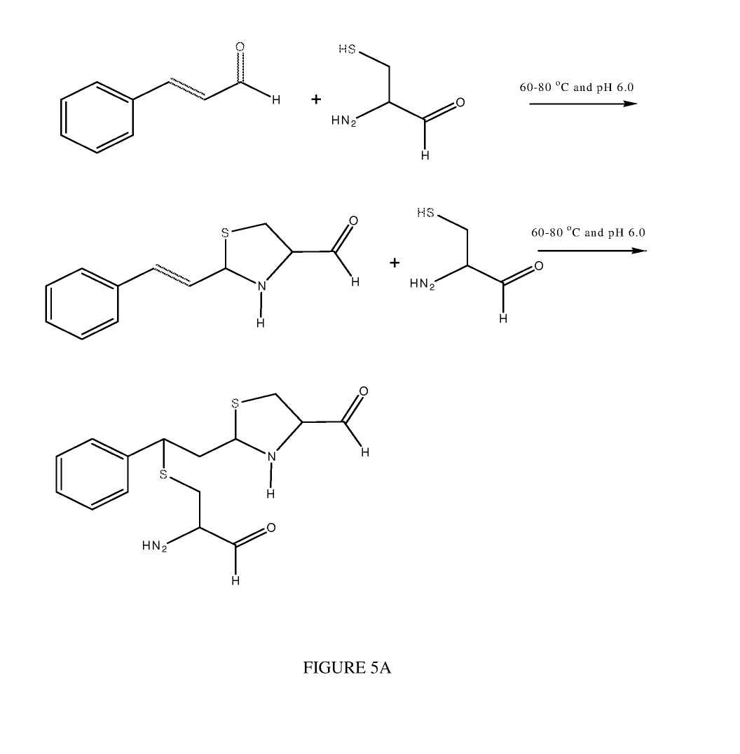

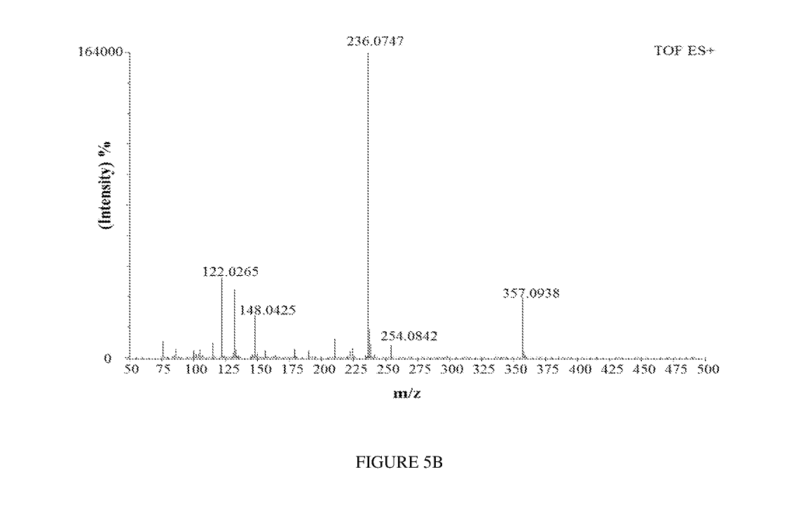

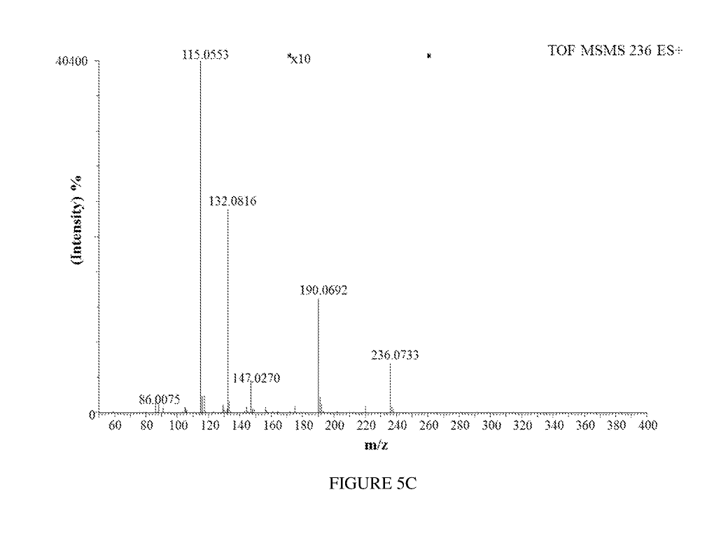

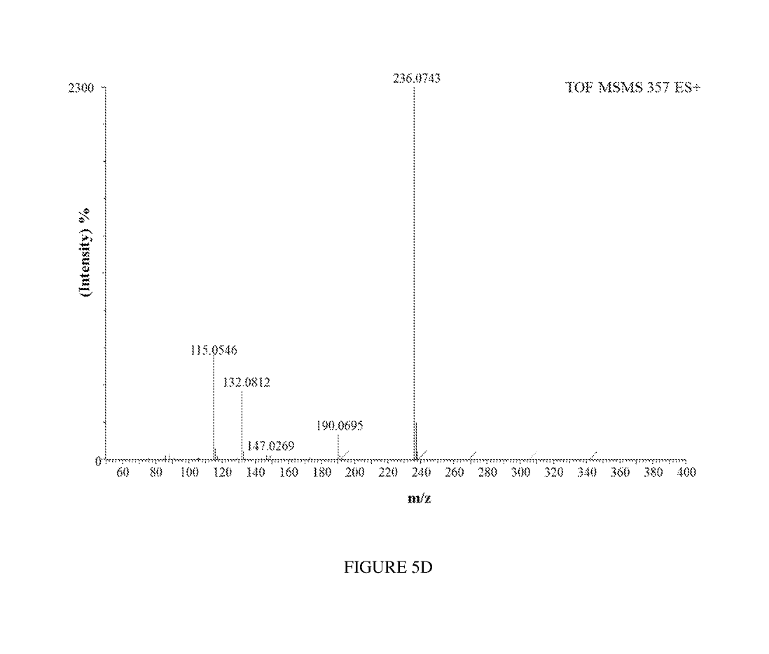

The potential reaction between cysteine and cinnamaldehyde (see FIG. 5A) was further investigated using Q-TOF LE/MS. Analyzing the detoxified products after reacting cysteine with cinnamaldehyde, two major peaks were presented at high intensities with 236.07 and 357.09 ions [M+1] (see FIG. 5B). An elemental composition analysis of these two revealed the potential formula of C₁₂H₁₃NO₂S and C₁₅H₂₀N₂O₄S₂, with mass error at less than 5 ppm. The first adduct product probably was a Schiff base (thiazolidine carboxylic acid), which can be formed by the condensation of cinnamaldehyde and cysteine. Previously, Michael addition has been also suggested as a reaction between thiol group of cysteine and α, β-unsaturated bond of cinnamaldehyde. However, the Michael addition product was not observed in the present detoxification process.

The second peak (357.09, [M+1]) probably was a diadduct, which can be formed the reaction of thiol group with between thiol group of cysteine and α, β-unsaturated bond of thiazolidine-4-carboxylic acid. To further confirm the reaction products, MS/MS was used to analyze these two major peaks (see FIGS. 5C and 5D). MS/MS spectrum showed four major fragments (115.06, 132.08, 147.03 and 190.07) from the precursor ion 236.07. The pathway could be explained thiazolidine derivative formation. It appeared that precursor ion lost COOH group to form ion 190.07, which could further lost C₂H₅N to form ion 147.03 or lost CH₂S to form ion 132.08.

Finally, the ion 115.05 was produced from the ion 147.03 by losing S or from ion 132.08 by losing NH₃. For the second product (357.09, [M+1]⁺), it appeared the precursor ion lost one cysteine (C₃H₇NSO₂) to form ion 236.07. The identified products further indicated that primary amine and thiol groups in cysteine played important roles in detoxifying cinnamaldehyde. The reaction of thiol group with carbonyl aldehyde to form hemithioacetal has been reported previously.

The amine group further reacts with hemithioacetal to form a five-membered thiazolidine ring. For glycine detoxification, the reaction of aldehydes and ketones with primary amine group in amino acids to form imine derivatives could be the main mechanism of detoxification. Therefore, Schiff base formation was likely the main reaction mechanism in detoxifying carbonyl aldehydes with nucleophilic amino acids (see Table 2).

TABLE 2
MS/MS data of products 1 and 2 from cysteine reaction with
cinnamaldehyde

| | Elemental | Exact mass | Calculated mass | |
|---|---|---|---|---|
| MS peaks | composition | [M + 1]⁺ | [M + 1]⁺ | ppm |
| Product 1 | C₁₂H₁₃NO₂S | 236.0745 | 236.0733 | 5.0 |
| Fragment 1-1 | C₉H₇ | 115.0548 | 115.0553 | 4.3 |
| Fragment 1-2 | C₉H₁₀N | 132.0813 | 132.0816 | 2.3 |
| Fragment 1-3 | C₉H₇S | 147.0268 | 147.0270 | 1.4 |
| Fragment 1-4 | C₁₁H₁₂NS | 190.0690 | 190.0692 | 1.1 |
| Product 2 | C₁₅H₂₀N₂O₄S₂ | 357.0943 | 357.0938 | 1.4 |
| Fragment 2-1 | C₁₂H₁₃NO₂S | 236.0745 | 236.0743 | 0.9 |
| Fragment 2-2 | C₉H₇ | 115.0548 | 115.0546 | 1.7 |
| Fragment 2-3 | C₉H₁₀N | 132.0813 | 132.0812 | 0.8 |
| Fragment 2-4 | C₁₁H₁₂NS | 190.0690 | 190.0695 | 2.6 |
| Fragment 2-5 | C₉H₇S | 147.0268 | 147.0269 | 0.7 |

Mass spectrum of detoxified hydrolysates with cysteine revealed that the potential reaction mechanism was probably related to the Schiff base formation by a condensation between carbonyl aldehyde (cinnamaldehyde) and nucleophilic amino acid (cysteine).

What is claimed is:

1. A method of fermenting a lignocellulosic biomass, the method comprising the steps of
pretreating the lignocellulosic biomass to provide a hydrolysate;
adding a nucleophile to the hydrolysate; and
adding a microorganism to the hydrolysate to produce an alcohol,
wherein a sufficient amount of the nucleophile is added to deactivate carbonyl-containing fermentation inhibitors in the hydrolysate.

2. The method of claim 1, wherein the hydrolysate is filtered into a solid fraction and a liquid fraction prior to addition of the nucleophile to the liquid fraction.

3. The method of claim 1, wherein the lignocellulosic biomass is selected from the group consisting of corn, corn stover, corn cobs, wood chips, softwood wood chips, hardwood wood chips, wheat straw, rice straw, hybrid poplar, sugarcane bagasse, switchgrass, miscanthus, forest thinnings, forest residues, agricultural residues, and combinations thereof.

4. The method of claim 1, wherein the pretreatment step comprises addition of an acid.

5. The method of claim 1, wherein the hydrolysate comprises sugars selected from the group consisting of glucose, mannose, xylose, galactose, arabinose, and combinations thereof.

6. The method of claim 1, wherein the hydrolysate is substantially not fermentable.

7. The method of claim 1, wherein the fermentation inhibitor is a ketone or an aldehyde.

8. The method of claim 1, wherein the addition of the nucleophile to the hydrolysate is performed prior to addition of the microorganism.

9. The method of claim 1, wherein the nucleophile is an amino acid.

10. The method of claim 1, wherein the nucleophile is cysteine, histidine, or a combination thereof.

11. The method of claim 1, wherein the nucleophile is cysteine.

12. The method of claim 11, wherein the concentration of cysteine is about 5.0 mM.

13. The method of claim 1, wherein the microorganism is Saccharomyces cerevisiae.

14. The method of claim 1, wherein the addition of the nucleophile to the hydrolysate is performed at a temperature of about 50° C. to about 90° C.

15. The method of claim 1, wherein the addition of the nucleophile to the hydrolysate is performed at a pH of about 6 to about 8.

16. The method of claim 1, wherein the alcohol is ethanol.

17. A method of fermenting a lignocellulosic biomass, the method comprising the steps of
pretreating the lignocellulosic biomass to provide a slurry;
adding a nucleophile to the slurry to remove fermentation inhibitors from the slurry; and
adding a microorganism to the slurry to produce an alcohol,
wherein a sufficient amount of the nucleophile is added to deactivate carbonyl-containing fermentation inhibitors in the slurry and
wherein the slurry is not separated into a solid fraction and a liquid fraction prior to addition of the nucleophile.

18. The method of claim 17, further comprising the step of adding one or more cellulases to the slurry resulting in hydrolysis of the slurry.

19. The method of claim 18, wherein the hydrolysis and the production of the alcohol are simultaneous.

20. The method of claim 17, wherein the pH of the slurry is adjusted prior to addition of the nucleophile.
